(19) United States
(12) Patent Application Publication
(10) Pub. No.: US 2009/0298813 A1
(43) Pub. Date: Dec. 3, 2009

(54) USE OF NEUROPROTECTIVE COMPOUNDS IN OBTAINING MEDICAMENTS INTENDED FOR THE TREATMENT OF NEURODEGENERATING DISEASES

(75) Inventors: Alain Le Ridant, Neuilly sur Seine (FR); Catherine Harpey, Paris (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, SURESNES (FR)

(21) Appl. No.: 12/448,719

(22) PCT Filed: Jan. 4, 2008
(86) PCT No.: PCT/FR2008/000014

§ 371 Date: Jul. 1, 2009

(30) Foreign Application Priority Data
Jan. 5, 2007 (FR) 0700048

(51) Int. Cl. A61K 31/4439; A61K 31/496; A61K 31/444; A61K 31/4545; A61K 31/551; A61P 25/00

(52) U.S. Cl. 514/219; 514/339; 514/253.09; 514/333; 514/318

(57) ABSTRACT

Use of neuroprotective compounds in obtaining medicaments intended for the curative treatment of neurodegenerative disease and/or the prevention of the appearance of disorders ensuing from those diseases.

The present invention relates to the use of neuroprotective compounds in obtaining medicaments intended for the treatment of neurodegenerative diseases.

With ageing of the population, the increase in neurodegenerative diseases is becoming a major public health problem, especially when they result in dementia and/or dependency.

The number of people aged 85 or more will quadruple between now and the year 2050. Worldwide, the number of people suffering from Alzheimer's disease, the most frequent of the neurodegenerative pathologies, is estimated to be 15 million. 75% of degenerative dementias are due to Alzheimer's disease and it causes 96% of the dementias in patients aged 85 and above.

The nervous system comprises several types of cell: the neuron, which is responsible for the transmission of the nerve impulse, and the glial cells, which occupy the space left vacant by the neurons. Unlike the cells of the liver or skin, neurons are not interchangeable. Each of them plays a particular part in the functioning of the whole system. Accordingly, different neurodegenerative diseases will affect nerve cells of a different type or location and result in a different symptomotology: Parkinson's disease is due to damage to dopaminergic neurons and the initial symptoms are motor symptoms; in multiple sclerosis, destruction of the myelin sheath, which is composed of oligodendrocytes, a variety of glial cell, causes disturbance of transmission of the nerve impulse, which gives rise to serious disturbances of motility, language and memory; in Alzheimer's disease, it is essentially the acetylcholine neurons of the hippocampus which are damaged, initially causing a deterioration in memory.

Neurodegenerative diseases therefore include amongst them different entities which are brought about by the destruction (most often of unknown cause) of different neurons and which will manifest themselves by different symptoms. In spite of the scientific advances of the last twenty years, treatment is still purely palliative, being administered in order to alleviate symptoms: levodopa and/or dopaminergic agonists in order to compensate for the lack of dopamine in Parkinson's disease; anticholinesterase agents in order to delay degradation of acetylcholine in Alzheimer's disease. However, symptomatic treatments, even if they do improve quality of life, do not prevent the inescapable progression of the disease due to continued neuronal death; it is now known that, when the first symptoms of Parkinson's disease appear, 60 to 80% of dopaminergic neurons have already been destroyed (Jankovic J. et al., Parkinson's Disease and Movement Disorders. Urban/Schwarzenberg, Baltimore, Md., (1988), 95-119); it is thought that the neurodegeneration of Alzheimer's disease starts 20 to 30 years before the clinical signs appear (Davies L. et al., Neurology, (1988), 38, 1688-1693). In the course of this early stage, which includes minor cognitive impairment (MCI), minor disturbances of memory appear, which should constitute a warning phase for early monitoring for possible progression towards Alzheimer's disease. The rate of conversion from MCI to Alzheimer's disease with clinical signs of dementia is from 10 to 15% per year (Petersen R. C., Journal of Internal Medicine, (2004), 256, 183-194; Visser P. J., Scheltens P., Verhey F. R., J Neurol Neurosurg Psychiatry, (2005), 76, 1348-1354). Unfortunately, there is currently no treatment which makes it possible to halt the neurodegeneration.

Accordingly, in 2003, a group of American experts (M. A. Dichter and R. E. Locke, Expert Opin. Emerging Drugs (2003), 8(1), 267-271) drew attention to the similarity of certain mechanisms common to the neuronal damage occurring during disparate, acute or chronic, neurodegenerative pathologies, including Alzheimer's disease, Parkinson's disease, amyotrophic lateral sclerosis, cerebral vascular accident, damage to the central nervous system following cardiac or neonatal surgery, epilepsy, cerebral or spinal cord trauma, and also certain neurodegenerative aspects encountered in psychiatric disorders.

The objective in considering the common mechanisms found in the course of neuronal death was to provide approaches for developing new therapeutic strategies. Indeed, when considering Alzheimer's disease (Sellal F., Nieoullon A., Michel G. et al., Thérapie, (2005), 60 (2), 89-107), Parkinson's disease (Rascol O., Goetz C., Koller W. et al., The Lancet, (2002), 359, 1589-1598; O. Rascol, Mouvements (2005), 1(1), 3-14) and Huntington's disease (Brusa L., Versace V., Koch G. et al., Annals of Neurology, (2005), 58 (4), 655-656), the experts repeatedly mention the limits of symptomatic treatment, which is the only current treatment, and the impossibility, hitherto, of delaying the progression of these diseases.

The point that is common to these neurodegenerative diseases is the process of neuronal death by apoptosis, or programmed cell death (Rao R. V., Bredesen D. E., Current Opinion in Cell Biology, (2004), 16, 653-662). This final phase can be triggered by functional impairment of various pathways (Bredesen D. E., Rao R. V., Mehlen P., Nature, (2006), 443, 796-802).

Apoptosis does not solely affect neurons. Astrocytes, which belong to the glial cells, were long considered to be simple support cells in the central nervous system. It is now well established that these cells also play a part in nourishing neurons, supplying them with the energy necessary to the activity of the nerve cells and regulating homeostasis of the extracellular medium in terms of ions and neurotransmitters by means of transport and reuptake systems. The astrocytes are active partners in synaptic transmission (Takuma K., Baba A., Matsuda T., Progress in Neurobiology, (2004), 72, 111-127). The astrocyte reactions of hypertrophy/hyperplasia (astrogliosis) and uncontrolled proliferation (astrocytosis) are observed in the course of pathologies as varied as viral infections, including HIV dementia, demyelinating inflammatory pathologies, cerebral traumas, cerebral ischaemia/hypoxia, multiple sclerosis, trisomy 21 and Alzheimer's disease (Takuma K., Baba A., Matsuda T., Progress in Neurobiology, (2004), 72, 111-127). Although the astrocyte reaction is considered to be beneficial in the early phase of lesional stress, making it possible to initiate repair of the extracellular matrix and release of neurotrophins necessary to the survival of the neurons, the toxic mediators produced by the activated astrocytes have a detrimental effect and are implicated in the pathogenesis of numerous neurodegenerative diseases (Aschner M., Neurotoxicology, (1998), 19, 269-281; Becher B., Prat A., Antel J. P., Glia, (2000), 29, 293-304).

Limiting astrogliosis and astrocytosis is therefore an objective necessary for neuron survival. Furthermore, however, astrocytosis is followed by apoptosis of the astrocytes, and the astrocytes in the course of disappearing are toxic to their surroundings (Lin J. H., Weigel H., Cotrina M. L. et al., Nature Neuroscience, (1998), 1 (6), 494-500). Preventing astrocyte apoptosis accordingly contributes to neuroprotection.

A new step was taken when it became possible to demonstrate, contrary to what had been believed, that neurogenesis also exists in adult humans (Eriksson P.S., Perfilieva E., Björk-Eriksson T. et al., Nature Medicine, (1998), 4 (11), 1313-1317). This discovery opens the way to a reparatory therapy in neurodegenerative diseases, wherein the first approach explored is the implanting of stem cells capable of resulting in mature neurons. However, it has also been discovered that a particular sub-population of astrocytes has retained stem cell and precursor cell properties, depending on the region (Noctor S. C., Flint A. C., Weissman T. A. et al., Nature, (2001), 409, 714-720). The possibility of using this natural store of endogenous stem cells that is brought about by adult neurogenesis by controlling the proliferation and differentiation of these cells appears an original and less invasive therapeutic approach. Stimulating endogenous sources of stem cells by administering differentiation factors would constitute a judicious alternative to the implanting of stem cells (Crespel A., Baldy-Moulinier M., Lerner Natoli M., Rev Neurol (Paris), (2004), 160 (12), 1150-1158; Freundlieb N., François C., Tandé D. et al., The Journal of Neuroscience, (2006), 26 (8), 2321-2325).

In slowly progressing neurodegenerative pathologies, the hypothesis of reduced physiological neurogenesis has been considered, especially for Alzheimer's disease (Zitnik G., Martin G. M., Journal of Neuroscience Research, (2002), 70, 258-263). Implanting embryonic stem cells has already been carried out in humans in Parkinson's disease (Bjorklund A., Dunnett S. B., Brundin P. et al., Lancet Neurol, (2003), 2, 437-445) and Huntington's chorea (Peschanski M., Dunnett S. B., Lancet Neurol, (2002), 1, 81), and amyotrophic lateral sclerosis (Silani V., Leigh N., Amyotroph Lateral Scler Other Motor Neuron Disord, (2003), 4, 8-10). However, stimulation of endogenous stem cells is only at the experimental stage in animals.

Whether the intention is to delay the progression of neurodegenerative diseases. (neuroprotection) or a fortiori to repair the lesions (neurogenesis), there currently exists no drug therapy application. The reasons for the setbacks and the need to discover new therapeutic approaches have recently been described for Parkinson's disease (Waldmeier P., Bozyczko-Coyne D., Williams M. et al., Biochemical Pharmacology, (2006), 72, 1197-1106).

More generally:
The major scientific advances relating to the molecular biological anomalies at the root of these diseases have not yet been translated into therapies. For example, the cause of Parkinson's disease remains unknown.
Therapeutic models do not exactly reproduce the human diseases. Accordingly, despite very promising results showing neuroprotection in animals, with a mechanism of action that is well delineated in molecular biology terms of reduction of apoptosis and lessening of microglial inflammation, CEP-1347 accelerated the progression of Parkinson's disease in clinical trials instead of delaying it (Shoulson I., Parkinson Study Group, PRECEPT Investigators, Neurology, (2006), 67, 185; Lund S., Porzgen P., Mortensen A. L., et al., Journal of Neurochemistry, (2005), 92, 1439-1451). Development of the product was suspended. With the creation of transgenic mice, a substantial advance was made as of 1995 in Alzheimer's disease, based on the identification of a certain number of gene mutations that are implicated in that disease. However, although these mice did allow advances to be made in respect of knowledge of the physiopathological pathways of Alzheimer's disease, assessing new pharmacological compounds is beset by the problem that these models reproduce only some of the neuropathological lesions or some of the cognitive impairments observed in Alzheimer's disease (Sellal F., Nieoullon A., Michel G. et al., Thérapie, (2005), 60 (2), 89-107). In particular, these models are able to reproduce the “amyloid” or “tauopathic” type lesions but are not automatically accompanied by neuronal death. For those pathologies that cannot be brought about by the transgenic route, the method of causation is artificial and does not exactly reproduce the human disease: administration of toxic agent (6-hydroxydopamine (6-OHDA) or methyl phenyltetrahydropyridine (MPTP)) for Parkinson's disease, standardised ligature or occlusion for simulating cerebral vascular accident, electric stimulation or glutamatergic agonists for causing epilepsy and the neuronal damage that follows (Dichter M. A., Locke R. E., Expert Opinion Emerging Drugs, (2003), 8 (1), 267-271).
Demonstrating a neuroprotective effect in a clinical context can be done only on the basis of indirect criteria, the validation of which is performed by correlation with clinical criteria. In Parkinson's disease, the first publications showing, in humans, striatal hypofunctioning by reducing the reuptake rate of 6-¹⁸-fluoro-1-dopa (FTD) by positron emission tomography (PET) date from 1996 (Morrish P. K., Sawle G. V., Brooks D. J., Brain, (1996), 119, 2097-2103). However, it was β-CIT (2β-carbomethoxy-3β-[4-iodophenyl]tropane) that made it really possible to evaluate the density of dopamine transporters (DATs), which directly reflects the functional integrity of the nigrostriatal system (Marek K., Innis R., van Dyck C. et al., Neurology, (2001), 57, 2089-2094). β-CIT (DaTSCAN) was approved by the European authorities in July 2000.
In Alzheimer's disease, the temporoparietal hypometabolism identified using the same technique (FTD-PET) makes it possible to differentiate Alzheimer's disease from other dementias and to predict the progression from MCI to Alzheimer's disease (Silverman D. H. S., Small G. W., Chang C. Y. et al., JAMA, (2001), 286 (17), 2120-2127; Minoshima S., Foster N. L., Sima A. A. et al., Annals of Neurology, (2001), 50, 358-365; Arnaiz E., Jelic V., Almkvist O. et al., Neuroreport, (2001), 12, 851-855; Chételat G., Desgranges B., de la Sayette V. et al., Neurology, (2003), 60, 1374-1377). Publications of the studies date from 2001.
Taking into account the role of astrocytes in maintaining neuron survival in neurodegenerative pathologies is recent (Takuma K., Baba A., Matsuda T., Progress in Neurobiology, (2004), 72, 111-127).
Even more so, the possibilities of neuron renewal are recent ideas (Eriksson P.S., Perfilieva E., Björk-Eriksson T. et al., Nature Medicine, (1998), 4 (11), 1313-1317) but have not yet been translated into clinical applications. The first attempts at injecting, into the damaged striatum, glial-line-derived neurotrophic factor (GNDF) growth factor in humans were suspended by the Amgen company in the absence of significant results in a Phase II study despite promising results in animals (Lang A. E., Gill S., Patel N. K. et al., Annals of Neurology, (2006), 59, 459-466). The invasive character of the administrations must be emphasised, an intracerebral catheter being fed by an abdominal pump.

All these new ideas, which are overturning the pharmacological approaches to neurodegenerative diseases, are manifesting themselves in the clinical context with the health authorities taking it upon themselves to revise the recommendations governing demonstration of the efficacy of a drug during clinical trials. Hitherto, these authorities considered that progression could be judged only on the basis of symptomatic criteria. Now they are recognising the new, neuroprotectively targeted therapeutic approaches. As an example thereof, in November 2005 the European Medicines Agency (EMEA) set up a working group for revision of the recommendations in Parkinson's disease. In-Alzheimer's disease, the working group created at the same date is to determine the means for ascertaining halting progression of the disease.

Accordingly, halting progression of neurodegenerative diseases and, even better, regeneration of the damaged cerebral zones still remains today a primary aim which has not yet been reached in order to check the increase in neurodegenerative diseases which is accompanying the ageing of the population.

Patent applications have been directed at original compounds for their tyrosine hydroxylase (TH)-inducing properties. It is known that TH is a rate-limiting enzyme which controls especially synthesis of the transmitter in central catecholaminergic and dopaminergic neurons. These compounds accordingly make it possible to compensate for the deficient synthesis of these neurotransmitters and to treat the symptoms associated with that deficiency.

With the advance in scientific knowledge and means of investigation, it has been shown, surprisingly, that some of these compounds have neuroprotective properties. They should accordingly be capable of halting the progression of neurodegenerative diseases, and indeed even of bringing about neurogenesis and thereby restoring the damaged cerebral zones.

The present invention relates more especially to the use of original compounds having neuroprotective properties which are accordingly useful in the treatment of Alzheimer's disease, earlier forms of dementia such as MCI, Parkinson's disease, Huntington's disease, multiple sclerosis, motor neuron diseases such as amyotrophic lateral sclerosis, pathological ageing, defects of cerebral perfusion such as cerebral vascular accident of thrombotic origin, haemorrhagic origin or following cardiac surgery, epilepsy, damage to the central nervous system following cardiac or neonatal surgery, epilepsy, cerebral or spinal cord trauma, and also certain neurodegenerative aspects of phenomena of neuroplasticity encountered in psychiatric disorders such as depression, schizophrenia, autism, dyslexia, senile dementias, virus diseases (HIV) or prion diseases, attention-deficit hyperactivity and also all pathologies of the white matter such as the lesions of periventricular leukomalacia of premature infants, atrophy of the cerebral white matter associated with ageing, with hypertension and leading to dementia, and the lesions of leukoaraiosis.

The compounds according to the invention are also useful in prevention of the appearance of disorders ensuing from neurodegenerative diseases.

The neuroprotective compounds according to the invention are more especially the compounds of formula (I) described in the Patent Application EP 0 658 557:

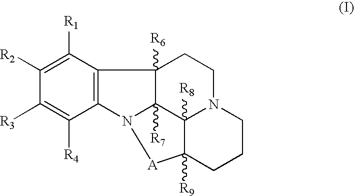

wherein:
R₁, R₂, R₃ and R₄, which may be identical or different, represent independently of one another a hydrogen or halogen atom, a hydroxy group,
a linear or branched (C₁-C₆)alkyl group optionally substituted by one or more halogen atoms or amino, nitro, linear or branched (C₁-C₆)alkoxy or optionally substituted aryl groups,
a linear or branched (C₁-C₆)alkoxy group optionally substituted by one or more halogen atoms or amino, nitro or linear or branched (C₁-C₆)alkoxy groups,
or R₁, R₂, R₃ and R₄, taken in pairs and carried by adjacent carbon atoms, form a methylenedioxy or ethylenedioxy group,
R₆ and R₇ each represent a hydrogen atom, adopting a cis-configuration with respect to one another; or they together form a bond,
R₈ and R₉ each represent a hydrogen atom, adopting a cis- or trans-configuration with respect to one another, or, when R₆ and R₇ together form a bond, they together form a bond,
A represents a bivalent radical

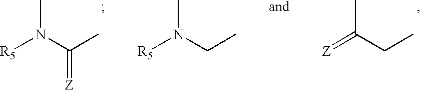

Z represents an oxygen or sulphur atom,
R₅ represents a hydrogen atom or a linear or branched (C₁-C₆)alkyl group optionally substituted by one or more halogen atoms or linear or branched (C₁-C₆)alkoxy groups, —NR₁₀R₁₁ groups, or phenyl groups optionally substituted by one or more halogen atoms, linear or branched (C₁-C₆)alkyl groups or linear or branched (C₁-C₆)alkoxy groups, these alkyl or alkoxy radicals being optionally substituted by one or more optionally substituted aryl groups,
R₁₀ and R₁₁, which may be identical or different, represent independently of one another a hydrogen atom or a linear or branched (C₁-C₆)alkyl or linear or branched (C₁-C₆)alkoxy group.

Even more preferably, the neuroprotective compounds of formula (I) according to the invention are:
(2RS,7SR),(3RS,16RS)-10-chloro-15-oxo-2,7,14,15-tetrahydro-14-aza-20,21-dinoreburnameninium chloride,
(2RS,7SR),(3RS,16RS)-10-chloro-14-methyl-2,7,14,15-tetrahydro-14-aza-20,21-dinoreburnamenin-15-one,
(2RS,7SR),(3RS,16RS)-14-benzyl-10-chloro-2,7,14,15-tetrahydro-14-aza-20,21-dinoreburnamenin-15-one,
(2RS,7SR),(3RS,16RS)-10-chloro-2,7,14,15-tetrahydro-14-aza-20,21-dinoreburnameninium dichloride,
(3RS,16RS)-10-chloro-14,15-dihydro-14-aza-20,21-dinoreburnamenin-15-one,
(2RS,7SR),(3RS,16RS)-15-oxo-2,7,14,15-tetrahydro-14-aza-20,21-dinoreburnameninium chloride,
(2RS,7SR),(3RS,16RS)-10-bromo-15-oxo-2,7,14,15-tetrahydro-14-aza-20,21-dinoreburnameninium chloride,
(2RS,7SR),(3RS,16RS)-10-methoxy-15-oxo-2,7,14,15-tetrahydro-14-aza-20,21-dinoreburnameninium chloride,
(2RS,7SR),(3RS,16RS)-11-methoxy-15-oxo-2,7,14,15-tetrahydro-14-aza-20,21-dinoreburnameninium chloride,
(2RS,7SR),(3RS,16RS)-10,11-dimethoxy-15-oxo-2,7,14,15-tetrahydro-4-aza-20,21-dinoreburnameninium chloride,
(2RS,7SR),(3RS,16RS)-10-trifluoromethoxy-2,7,14,15-tetrahydro-14-aza-20,21-dinoreburnamenin-15-one,
(2RS,7SR),(3RS,16RS)-10,11-methylenedioxy-15-oxo-2,7,14,15-tetrahydro-14-aza-20,21-dinoreburnameninium chloride,
(2RS,7SR),(3RS,16RS)-10-methyl-15-oxo-2,7,14,15-tetrahydro-14-aza-20,21-dinoreburnameninium chloride,
(2RS,7SR),(3RS,16RS)-11-methyl-15-oxo-2,7,14,15-tetrahydro-14-aza-20,21-dinoreburnameninium chloride,
(2RS,7SR),(3RS,16RS)-10-chloro-2,7,14,15-tetrahydro-14-aza-20,21-dinoreburnamenine-15-thione,
14,15-dihydro-3,16-didehydro-14-aza-20,21-dinorebumamenin-15-one,
trans-(3RS,16RS)-14,15-dihydro-14-aza-20,21-dinorebumamenin-15-one,
trans-(3RS,16RS)-14,15-dihydro-14-aza-20,21-dinoreburnameninium dichloride,
(2SR,7RS),(3RS,16RS)-2,7, 14,15-tetrahydro-14-aza-20,21-dinoreburnameninium dichloride,
(1 aRS,12bRS),(7aSR,12aRS)-9-chloro-1a,2,3,6,7,7α,12a,12b-octahydro-1H,4H-benzo[5,6]pyrrolizino[2,1,7-ija]quinolizin-1-one,
(1 aRS,12bRS),(7aSR,12aRS)-9-methyl-1a,2,3,6,7,7a,12a,12b-octahydro-1H,4H-benzo[5,6]pyrrolizino[2,1,7-ija]quinolizin-1-one,
(1 aRS,12bRS),(7aSR,12aRS)-9-methoxy-1a,2,3,6,7,7a,12a,12b-octahydro-1H,4H-benzo[5,6]pyrrolizino[2, 1 ,7-ija]quinolizin-1-one,
(2RS,7SR),(3RS,16RS)-14-benzyl-2,7,14,15-tetrahydro-14-aza-20,21-dinoreburnamenin-15-one,
trans-(3RS,16RS)-14-benzyl-14,15-dihydro-14-aza-20,21-dinoreburnamenin-15-one,
   their enantiomers and diastereoisomers, and also addition salts thereof with a pharmaceutically acceptable acid or base.

In even more preferable manner, a neuroprotective compound of formula (I) according to the invention is (+)-(2RS,7SR),(3RS,16RS)-10-chloro-15-oxo-2,7,14,15-tetrahydro-14-aza-20,21-dinoreburnameninium chloride, and also addition salts thereof with a pharmaceutically acceptable acid or base.

Other neuroprotective compounds according to the invention are, more especially, compounds of formula (II):

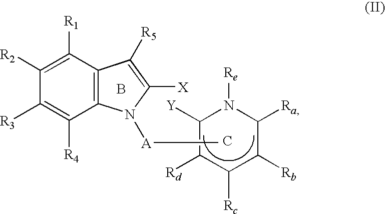

wherein:
A represents a divalent radical:

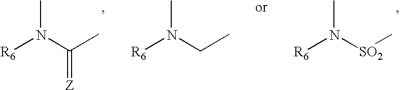

wherein:
   Z represents an oxygen atom or sulphur atom,
   R₆ represents a hydrogen atom, a linear or branched (C₁-C₆)alkyl group, C(O)-AA wherein AA represents an amino acid radical, a linear or branched (C₁-C₆)alkoxy-carbonyl group, CHR′—O—C(O)—R″ wherein R′ represents a hydrogen atom or a linear or branched (C₁-C₆)alkyl group and R″ represents a linear or branched (C₁-C₆)alkyl group, a linear or branched (C₂-C₆)alkenyl group, an aryl group, an aryl-(C₁-C₆)alkyl group in which the alkyl moiety is linear or branched, a linear or branched (C₁-C₆)polyhaloalkyl group, or a linear or branched (C₁-C₆)alkyl chain substituted by one or more halogen atoms, one or more hydroxy groups, linear or branched (C₁-C₆)alkoxy groups, or amino groups optionally substituted by one or two identical or different, linear or branched (C₁-C₆)alkyl groups,
in the ring B
[symbol] represents a single bond or a double bond,
in the ring C
[symbol] represents a single bond or a double bond, the ring C containing, at most, only one double bond,
R₁, R₂, R₃ and R₄, which may be the same or different, each independently of the others, represent a hydrogen or halogen atom, a linear or branched (C₁-C₆)alkyl group, a linear or branched (C₁-C₆)alkoxy group, a hydroxy group, a cyano group, a nitro group, a linear or branched (C₁-C₆)polyhaloalkyl group, an amino group (optionally substituted by one or two linear or branched (C₁-C₆)alkyl and/or linear or branched (C₂-C₆)alkenyl groups, it being possible for the alkyl and alkenyl groups to be the same or different), or a linear or branched (C₁-C₆)alkyl chain substituted by one or more halogen atoms, one or more hydroxy groups, linear or branched (C₁-C₆)alkoxy groups, or amino groups optionally substituted by one or two identical or different, linear or branched (C₁-C₆)alkyl groups,
R₅ represents a hydrogen atom, a linear or branched (C₁-C₆)alkyl group, an aminoalkyl group in which the alkyl moiety is a linear or branched chain of 1 to 6 carbon atoms, or a linear or branched (C₁-C₆)hydroxyalkyl group,
X and Y, which may be the same or different, each independently of the other, represent a hydrogen atom or a linear or branched (C₁-C₆)alkyl group,
Rₐ, R_{b}, R_{c} and R_{d}, which may be the same or different, each independently of the others, represent a hydrogen or halogen atom, a linear or branched (C₁-C₆)alkyl group, a hydroxy group, a linear or branched (C₁-C₆)alkoxy group, a cyano group, a nitro group, a linear or branched (C₁-C₆)polyhaloalkyl group, an amino group (optionally substituted by one or two identical or different, linear or branched (C₁-C₆)alkyl groups), or a linear or branched (C₁-C₆)alkyl chain substituted by one or more groups selected from halogen, hydroxy, linear or branched (C₁-C₆)alkoxy, and amino optionally substituted by one or two identical or different, linear or branched (C₁-C₆)alkyl groups,
it being understood that when A is linked to the ring C at a carbon atom carrying one of the substituents Rₐ, R_{b}, R_{c}, R_{d} or Y and said linking carbon atom also carries a double bond, then the corresponding substituent Rₐ, R_{b}, Rₐ, R_{d} or Y is absent,
R_{c} represents a hydrogen atom, a linear or branched (C₁-C₆)alkyl group; an aryl-(C₁-C₆)alkyl group in which the alkyl moiety is linear or branched; a linear or branched (C₂-C₆)alkenyl group; a linear or branched (C₂-C₆)alkynyl group; a linear or branched (C₁-C₆)alkyl chain substituted by one or more groups selected from hydroxy, amino (optionally substituted by one or two identical or different, linear or branched (C₁-C₆)alkyl groups), linear or branched (C₁-C₆)alkoxy, and NR₇R₈ wherein R₇ and R₈, together with the nitrogen atom carrying them, form an optionally substituted, 4- to 8-membered heterocycle optionally containing one or more double bonds within the heterocycle and optionally containing within the cyclic system a second hetero atom selected from an oxygen atom and a nitrogen atom; or a linear or branched (C₂-C₆)alkenyl chain substituted by the same groups as the alkyl chain or a linear or branched (C₂-C₆)alkynyl chain substituted by the same groups as the alkyl chain.

Even more preferably, the neuroprotective compounds of formula (II) according to the invention are:
N-(1H-indol-1-yl)-1-methyl-1,2,5,6-tetrahydropyridine-3-carboxamide,
N-(1H-indol-1-yl)-1-methyl-1,2,3,6-tetrahydropyridine-3-carboxamide,
N-(1H-indol-1-yl)-1-methyl-1,4,5,6-tetrahydropyridine-3-carboxamide,
N-(2,3-dihydro-1H-indol-1-yl)-1-methyl-1,4,5,6-tetrahydropyridine-3-carboxamide,
1-methyl-N-(2-methyl-2,3-dihydro-1H-indol-1-yl)-1,2,5,6-tetrahydropyridine-3-carboxamide,
N-(5-chloro-1H-indol-1-yl)-1-methyl-1,2,5,6-tetrahydropyridine-3-carboxamide,
N-(5-fluoro-1H-indol-1-yl)-1-methyl-1,2,5,6-tetrahydropyridine-3-carboxamide,
N-(5-methoxy-1H-indol-1-yl)-1-methyl-1,2,5,6-tetrahydropyridine-3-carboxamide,
N-(2,3-dimethyl-1H-indol-1-yl)-1-methyl-1,2,5,6-tetrahydropyridine-3-carboxamide,
N-(2,3-dimethyl-1H-indol-1-yl)-1-methyl-1,2,5,6-tetrahydropyridine-3-carboxamide,
N-(1H-indol-1-yl)-1-methyl-1,2,5,6-tetrahydropyridine-4-carboxamide,
1-allyl-N-(5-chloro-1H-indol-1-yl)-piperidine-3-carboxamide,
N-(5-chloro-1H-indol-1-yl)-piperidine-3-carboxamide,
3-[(1H-indol-1-ylamino)carbonyl]-1-methylpiperidinium chloride,
N-(2,3-dihydro-1H-indol-1-yl)-1-methyl-1,2,5,6-tetrahydro-pyridine-3-carboxamide,
1-methyl-N-(2-methyl-2,3-dihydro-1H-indol-1-yl)-piperidine-3-carboxamide,
N-(5-chloro-1H-indol-1-yl)-1-propyl-piperidine-3-carboxamide,
N-(5-chloro-1H-indol-1-yl)-1-(2-hydroxyethyl)-1,2,5,6-tetrahydro-3-pyridinecarboxamide,
1-[2-(dimethylamino)ethyl]-N-(1H-indol-1-yl)-1,2,5,6-tetrahydro-3-pyridinecarboxamide,
N-(5-chloro-1H-indol-1-yl)-1-[2-(dimethylamino)ethyl]-1,4,5,6-tetrahydro-3-pyridinecarboxamide,
1-[2-(dimethylamino)ethyl]-N-(1H-indol-1-yl)-1,4,5,6-tetrahydro-3-pyridinecarboxamide,
1-[2-(dimethylamino)ethyl]-N-(1H-indol-1-yl)-3-piperidinecarboxamide,
N-(1H-indol-1-yl)-1-[2-(4-morpholinyl)ethyl]-3-piperidinecarboxamide,
N-(1H-indol-1-yl)-1-[2-(1-piperidinyl)ethyl]-3-piperidinecarboxamide,
N-(1H-indol-1-yl)-1-[2-(4-methyl-1-piperazinyl)ethyl]-3-piperidinecarboxamide,
1-{2-[4-(2-Hydroxyethyl)-1-piperazinyl]ethyl}-N-(1H-indol-1-yl)-3′-piperidinecarboxamide,
N-(1H-indol-1-yl)-1-[2-(1-pyrrolidinyl)ethyl]-3-piperidinecarboxamide,
1-[2-(1-azepanyl)ethyl]-N-(1H-indol-1-yl)-3-piperidinecarboxamide,
N-(1H-indol-1-yl)-1-[2-(4-phenyl-1-piperazinyl)-ethyl]-3-piperidinecarboxamide trihydrochloride,
N-(1H-indol-1-yl)-N-({1-[2-(1-piperidinyl)ethyl]-3-piperidinyl} methyl)amine trihydrochloride,
N-(5-chloro-2,3-dihydro-1H-indol-1-yl)-1-(2-hydroxyethyl)-1,4,5,6-tetrahydro-3-pyridinecarboxamide,
N-(2,3-dihydro-1H-indol-1-yl)-1-[2-(dimethylamino)ethyl]-3-piperidinecarboxamide dihydrochloride,
N-(5-chloro-1H-indol-1-yl)-1-(2-hydroxyethyl)-1,4,5,6-tetrahydro-3-pyridinecarboxamide,
1-(2-hydroxyethyl)-N-(1H-indol-1-yl)-1,4,5,6-tetrahydro-3-pyridinecarboxamide,
N-(indol-1-yl)-N-[(1-methyl-1,2,5,6-tetrahydropyridin-3-yl)methyl]amine dihydrochloride,
1-benzyl-N-(5-chloro-1H-indol-1-yl)-3-piperidinecarboxamide,
3-{[(5-chloro-1H-indol-1-yl)amino]carbonyl}-1-methylpiperidinium hydrochloride,
(3R,4S)-3-(4-fluorophenyl)-N-(1H-indol-1-yl)-1-methylpiperidine-4-carboxamide,
(3R,4R)-3-(4-fluorophenyl)-N-(1H-indol-1-yl)-1-methylpiperidine-4-carboxamide,
tert-butyl 4-(2-{3-[(1H-indol-1-ylamino)carbonyl]-1-piperidinyl}ethyl)-piperazine-1-carboxylate,
1-[3-(dimethylammonium)propyl]-3-[(1H-indol-1-ylamino)carbonyl]piperidinium dihydrochloride,
N-(1H-indol-1-yl)-1-[3-(1-piperidinyl)propyl]-3-piperidinecarboxamide,
N-(1H-indol-1-yl)-1-[3-(4-methyl-1-piperazinyl)propyl]-3-piperidinecarboxamide,
N-(5-chloro-1H-indol-1-yl)-1-(2-hydroxyethyl)-3-piperidinecarboxamide,
1-(3-hydroxypropyl)-N-(1H-indol-1-yl)-3-piperidinecarboxamide,
N-(indol-1-yl)-1-[2-(4-methylpiperazin-1-yl)ethyl]-1,2,5,6-tetrahydropyridine-3-carboxamide,
N-(5-chloroindol-1-yl)-1-[2-(4-methylpiperazin-1-yl)ethyl]-1,2,5,6-tetrahydropyridine-3-carboxamide,
N-(5-methoxyindol-1-yl)-1-[2-(4-methylpiperazin-1-yl)ethyl]-1,2,5,6-tetrahydropyridine-3-carboxamide,
N-(5-methoxylindol-1-yl)-1-[2-(4-methylpiperazin-1-yl)ethyl]-1,2,5,6-tetrahydropyridine-5-carboxamide,
N-(5-methylindol-1-yl)-1-[2-(4-methylpiperazin-1-yl)ethyl]-1,2,5,6-tetrahydropyridine-3-carboxamide,
N-(5-methylindol-1-yl)-1-[2-(4-methylpiperazin-1-yl)ethyl]-1,2,5,6-tetrahydropyridine-5-carboxamide,
N-(indol-1-yl)-1-[2-(4-(2-hydroxyethyl)piperazin-1-yl)ethyl]-1,2,5,6-tetrahydropyridine-3-carboxamide,
N-(indol-1-yl)-1-(2-piperidin-1-yl-ethyl)-1,2,5,6-tetrahydropyridine-3-carboxamide,
N-(indol-1-yl)-1-[2-[3-(ethoxycarbonyl)-1,2,5,6-tetrahydropyridin-1-yl]ethyl]-1,2,5,6-tetrahydropyridine-3-carboxamide,
(±)-N-(indol-1-yl)-1-[2-[4-[(tetrahydrofuran-2-yl)methyl]piperazin-1-yl)]ethyl] piperidine-3-carboxamidyl trihydrochloride,
ethyl (±)-N-(indol-1-yl)-1-[2-[4-[2-(dimethylamino)ethyl]piperazin-1-yl)]ethyl]-piperidine-3-carboxylate tetrahydrochloride,
(±)-N-(indol-1-yl)-1-[2-[4-(2-methoxyethyl)piperazin-1-yl)]ethyl]piperidine-3-carboxamide trihydrochloride,
(±)-N-(indol-1-yl)-1-[2-[4-(1-methylpiperidin-4-yl)piperazin-1-yl)]ethyl]piperidine-3-carboxamide tetrahydrochloride,
(±)-N-(indol-1-yl)-1-[3-[4-(2-hydroxyethyl)piperazin-1-yl)]propyl]piperidine-3-carboxamide trihydrochloride,
(±)-N-(indol-1-yl) 1-[4-(4-methylpiperazin-1-yl)butyl]piperidine-3-carboxamide trihydrochloride,
(±)-N-(indol-1-yl)-1-[4-[4-(2-hydroxyethyl)piperazin-1-yl)]butyl]piperidine-3-carboxamide trihydrochloride,
(±)-N-(indol-1-yl)-1-[2-(4-butylpiperazin-1-yl)ethyl]piperidine-3-carboxamide trihydrochloride,
(±)-N-(indol-1-yl)-1-allylpiperidine-3-carboxamide,
(±)-N-(indol-1-yl)-1-(prop-2-ynyl)piperidine-3-carboxamide,
(±)-N-(indol-1-yl)-1-[4-(piperidin-1-yl)but-2-en-1-yl]-piperidine-3-carboxamide,
(R or S) (−)-N-(indol-1-yl)-1-[2-(piperidin-1-yl)ethyl]piperidine-3-carboxamide enantiomer 1,
(R or S) (+)-N-(indol-1-yl)-1-[2-(piperidin-1-yl)ethyl]piperidine-3-carboxamide enantiomer 2,
   their enantiomers and diastereoisomers, and also addition salts thereof with a pharmaceutically acceptable acid or base.

In even more preferable manner, the neuroprotective compounds of formula (II) according to the invention are N-(1H-indol-1-yl)-1-methyl-1,2,5,6-tetrahydropyridine-3-carboxamide and N-(5-chloroindol-1-yl)-1-[2-(4-methylpiperazin-1-yl)ethyl]-1,2,5,6-tetrahydropyridine-3-carboxamide, and also addition salts thereof with a pharmaceutically acceptable acid or base.

Other neuroprotective compounds according to the invention are, more especially, compounds of formula (III):

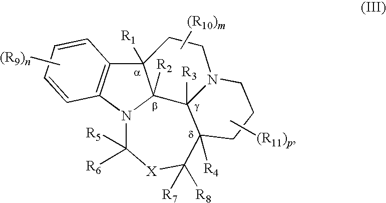

wherein:
R₁ represents a hydrogen atom or a linear or branched (C₁-C₆)alkyl group, a linear or branched (C₁-C₆)aminoalkyl group or a linear or branched (C₁-C₆)hydroxyalkyl group,
R₂ represents a hydrogen atom, or R₁ and R₂, together with the carbon atoms carrying them, form a carbon-carbon bond,
R₃ represents a hydrogen atom,
R₄ represents a hydrogen atom or a methyl or linear or branched (C₃-C₆)alkyl group, a linear or branched (C₁-C₆)aminoalkyl group, a linear or branched (C₁-C₆)hydroxyalkyl group, an aryl-(C₁-C₆)alkyl group in which the alkyl moiety is linear or branched, or a heterocycloalkyl-(C₁-C₆)alkyl group in which the alkyl moiety is linear or branched,
or R₃ and R₄, together with the carbon atoms carrying them, form a carbon-carbon bond,
R₅, R₆, R₇ and R₈, which may be identical or different, represent, each independently of the others, a hydrogen atom or a pair of geminal substituents (R₅ and R₆ and/or R₇ and R₈) form an oxo, thioxo or imino group,
R₉ represents a hydrogen or halogen atom or an optionally substituted, linear or branched (C₁-C₆)alkyl group, a linear or branched (C₁-C₆)alkoxy group, a hydroxy group, a cyano group, a nitro group, a linear or branched (C₁-C₆)polyhaloalkyl group or an amino group (optionally substituted by one or two linear or branched (C₁-C₆)alkyl(s), linear or branched (C₂-C₆)alkenyl(s), it being possible for the alkyls and alkenyls to be identical or different),
R₁₀ and R₁₁, which may be the same or different, represent, each independently of the other, a hydrogen or halogen atom or a linear or branched (C₁-C₆)alkyl group, a linear or branched (C₁-C₆)alkoxy group, a hydroxy group, a cyano group, a nitro group, a linear or branched (C₁-C₆)polyhaloalkyl group or an amino group (optionally substituted by one or two linear or branched (C₁-C₆)alkyl(s), linear or branched (C₂-C₆)alkenyl(s), it being possible for the alkyls and alkenyls to be identical or different),
n represents an integer between 0 and 4 inclusive (0, 1, 2, 3 or 4),
m represents an integer between 0 and 2 inclusive (0, 1 or 2),
p represents an integer between 0 and 3 inclusive (0, 1, 2 or 3),
X represents a group NR₁₂,
R₁₂ represents a hydrogen. atom or an optionally substituted, linear or branched (C₁-C₆)alkyl group, an optionally substituted, linear or branched (C₂-C₆)alkenyl group, an aryl-(C₁-C₆)alkyl group in which the alkyl moiety is linear or branched, or a linear or branched (C₁-C₆)polyhaloalkyl group.

Even more preferably, the neuroprotective compounds of formula (III) according to the invention are:
(5aRS,12aSR,12bSR,12cSR)-7-chloro-2,3,4,5,5a,10,11,12a,12b,12c-decahydro-1H,12H-3a,9b,11-triazabenzo[a]naphtho[2,1,8-cde]azulen-12-one,
(5aRS,12aSR,12bSR,12cSR)-7-chloro-2,3,5a,12a,12b,12c-hexahydro-1H,4H-3a,9b,11-triazabenzo[a]naphtho[2,1,8-cde]azulene-10,12(5H,11H)-dione,
(12aRS,12bRS)-7-chloro-2,3,12a,12b-tetrahydro-1H,4H-3a,9b,11-triazabenzo[a]-naphtho[2,1,8-cde]azulene-10,12(5H,11H)-dione,
(5aRS,12aSR,12bSR,12cSR)-2,3,5a,12a,12b,12c-hexahydro-1H,4H-3a,9b,11-triazabenzo[a]naphtho[2,1,8-cde]azulene-10,12(5H,11H)-dione,
(5aS,12aR,12bR,12cR)- or (5aR,12aS,12bS,12cS)-2,3,5a,12a,12b,12c-hexahydro-1H,4H-3a,9b,11-triazabenzo[a]naphtho[2,1,8-cde]azulene-10,12(5H,11H)-dione (enantiomer α),
(5aS,12aR,12bR,12cR)- or (5aR,12aS,12bS,12cS)-2,3,5a,12a,12b,12c-hexahydro-1H,4H-3a,9b,11-triazabenzo[a]naphtho[2,1,8-cde]azulene-10,12(5H,11H)-dione (enantiomer α),
(12aRS,12bRS)-2,3,12a,12b-tetrahydro-1H,4H-3a,9b,11-triazabenzo[a]naphtho[2,1,8-cde]azulene-10,12(5H,11H)-dione,
   their enantiomers and diastereoisomers, and also addition salts thereof with a pharmaceutically acceptable acid or base.

In even more preferable manner, the neuroprotective compounds of formula (III) according to the invention are (5aS,12aR,12bR,12cR)- or (5aR,12aS,12bS,12cS)-2,3 ,5a,12a,12b,12c-hexahydro-1H,4H-3a,9b,11-triazabenzo[a]naphtho[2, 1 ,8-cde]azulene-10,12(5H,11H)-dione, and also addition salts thereof with a pharmaceutically acceptable acid or base.

Other neuroprotective compounds according to the invention are, more especially, compounds of formula (IV):

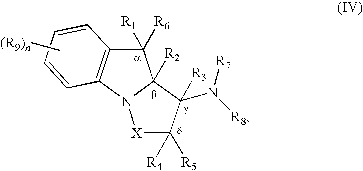

wherein:
X represents CO or

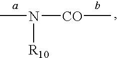

R₁ represents hydrogen, linear or branched (C₁-C₆)alkyl, linear or branched (C₁-C₆)aminoalkyl, linear or branched (C₁-C₆)hydroxyalkyl, aryl-(C₁-C₆)alkyl in which the alkyl moiety may be linear or branched,
R₂ represents hydrogen, linear or branched (C₁-C₆)alkyl, linear or branched (C₁-C₆)aminoalkyl, linear or branched (C₁-C₆)hydroxyalkyl,
or R₁ and R₂, together with the carbon atoms carrying them, form a carbon-carbon bond,
R₃ represents hydrogen, linear or branched (C₁-C₆)alkyl, linear or branched (C₁-C₆)aminoalkyl, linear or branched (C₁-C₆)hydroxyalkyl,
R₄ represents hydrogen, linear or branched (C₁-C₆)alkyl, linear or branched (C₁-C₆)aminoalkyl, linear or branched (C₁-C₆)hydroxyalkyl,
or R₃ and R₄, together with the carbon atoms carrying them, form a carbon-carbon bond,
R₅ represents hydrogen, linear or branched (C₁-C₆)alkyl,
R₆ represents hydrogen, linear or branched (C₁-C₆)alkyl,
R₇, R₈ represent a hydrogen atom, a group linear or branched (C₁-C₆)alkyl, aryl-(C₁-C₆)alkyl in which the alkyl moiety may be linear or branched, linear or branched (C₂-C₆)alkenyl, linear or branched (C₂-C₆)alkynyl, a linear or branched (C₁-C₆)alkyl chain substituted by one or more hydroxy, cyano, linear or branched (C₁-C₆)alkoxy, NR₁₃R₁₄, a linear or branched (C₁-C₆)alkenyl chain substituted by the same substituents as those defined for the alkyl chain, or a linear or branched (C₁-C₆)alkynyl chain substituted by the same substituents as those defined for the alkyl chain,
or,
either R₅ and R₈, together with the carbon and nitrogen atoms carrying them, form a heterocycle having 5, 6 or 7 ring members, optionally substituted by a group R₁₂,
or R₆ and R₇, together with the carbon and nitrogen atoms carrying them, form a heterocycle having 5, 6 or 7 ring members, optionally substituted by a group R₁₁,
it being understood that of necessity one, but only one, of the two groupings “R₅ and R₈” or “R₆ and R₇” together with the carbon and nitrogen atoms carrying them form a heterocycle having 5, 6 or 7 ring members, optionally substituted by a group R₁₁,
R₉ represents hydrogen, halogen, linear or branched (C₁-C₆)alkyl, linear or branched (C₁-C₆)alkoxy, hydroxy, cyano, nitro, linear or branched (C₁-C₆)polyhaloalkyl, NR₁₅R₁₆, or a linear or branched (C₁-C₆)alkyl chain substituted by one or more halogens, one or more hydroxy, linear or branched (C₁-C₆)alkoxy, or NR₁₅R₁₆,
n represents an integer 0, 1, 2, 3 or 4,
R₁₀ represents hydrogen, linear or branched (C₁-C₆)alkyl, linear or branched (C₂-C₆)alkenyl, aryl-(C₁-C₆)alkyl in which the alkyl moiety may be linear or branched, linear or branched (C₁-C₆)polyhaloalkyl, or a linear or branched (C₁-C₆)alkyl chain substituted by one or more halogen atoms, one or more groups hydroxy, linear or branched (C₁-C₆)alkoxy, or NR₁₅R₁₆,
R₁₁, R₁₂, which may be identical or different, represent a —COOT or —CH₂O-U group wherein T and U, which may be identical or different, represent a hydrogen atom or a linear or branched (C₁-C₆)alkyl group,
R₁₃, R₁₄, which may be identical or different, represent a hydrogen atom or a linear or branched (C₁-C₆)alkyl group or, together with the nitrogen atom carrying them, form an optionally substituted heterocycle having from 4 to 8 ring members optionally containing a double bond in the heterocycle and optionally containing in the ring system a second hetero atom selected from an oxygen atom and a nitrogen atom,
R₁₅, R₁₆, which may be identical or different, each represent a hydrogen atom or a group linear or branched (C₁-C₆)alkyl, linear or branched (C₂-C₆)alkenyl,
   it being understood that:
(3aSR,4SR)-3-benzyl-4-ethyl-2,3,3a,4-tetrahydrobenzo[b]pyrido[2,3,4-gh]pyrrolizin-5(1H)-one does not form part of the invention.

Even more preferably, the neuroprotective compounds of formula (IV) according to the invention are:
(4aSR,11 aSR,11bRS)-1-methyl-1,2,3,4,4a,11,11a,11b-octahydropyrido[2′,3′:3,4]-pyrrolo[1,2-a]indol-5-one,
(4aSR,11aRS,11bSR)-1-allyl-1,2,3,4,4a,11,11a,11b-octahydropyrido[2′,3′:3,4]pyrrolo-[1,2-a]indol-5-one,
(4aSR,11aSR,11bRS)-1-methyl-1,2,3,4,4a,11,11a,11b-octahydropyrido[2′,3′:3,4]-pyrrolo[1,2-a]indol-5-one,
(4aSR,11aSR,11bRS)-1,2,3,4,4a,11,11a,11b-octahydro-1,6,6a-triaza-benzo[a]fluoren-5-one,
(4aSR,11aSR,11bRS)-(5-oxo-3,4,4a,11,11a,1b-hexahydro-2H,5H-pyrido[2′,3′:3,4]-pyrrolo[1,2-a]indol-1-yl)acetonitrile,
(3aSR,6aRS,10cRS)-4-propyl-(3a,4,5,6,6a,10c-hexahydro)-3H-1,4,10b-triazafluor-anthen-2-one,
(3aRS,6aSR,10cRS)-4-propyl-(3a,4,5,6,6a,10c-hexahydro)-3H-1,4,10b-triazafluoranthen-2-one,
(4aSR,11aRS,11bSR)-1-allyl-9-methoxy-1,2,3,4,4a,11,11a,11b-octahydropyrido-[2′,3′:3,4]pyrrolo[1,2-a]indol-5-one,
(4aSR,11aSR,11bRS)-9-fluoro-1,2,3,4,4a,11,11a,11b-octahydropyrido[2′,3′:3,4]-pyrrolo[1,2-a]indol-5-one,
(4aSR,11aSR,11bRS)-9-chloro-1-methyl-1,2,3,4,4a,11,11a,11b-octahydropyrido-[2′,3′:3,4]pyrrolo[1,2-a]indol-,5-one,
1,9-dimethyl-1,2,3,4-tetrahydropyrido[2′,3′:3,4]pyrrolo[1,2-a]indol-5-one,
(11aRS)-1,9-dimethyl-1,2,3,4,11,11a-hexahydropyrido[2′,3′:3,4]pyrrolo[1,2-a]indol-5-one,
(4aS,11aR,11bS)-1-allyl-1,2,3,4,4a,11,11a,11b-octahydropyrido[2′,3′:3,4]pyrrolo[1,2-a]indol-5-one,
(4aR,11aS,11bR)-1-allyl-1,2,3,4,4a,11,11a,11b-octahydropyrido[2′,3′:3,4]pyrrolo[1,2-a]indol-5-one,
(3aRS,10bSR,10cRS)-3-benzyl-2,3,3a,4,10b,10c-hexahydrobenzo[b]pyrido[2,3,4-gh]-pyrrolizin-5(1H)-one,
(4aSR,11 aSR,11bRS)-1-allyl-1,2,3,4,4a,11,11a,11b-octahydropyrido[2′,3′:3,4]-pyrrolo[1,2-a]indol-5-one,
(4aS,11aS,11bR)-1-methyl-1,2,3,4,4a,11,11a,11b-octahydropyrido[2′,3′:3,4]pyrrolo[1,2-a]indol-5-one,
(4aSR,11 aRS,11bSR)-1,2,3,4,4a,11,11a,11b-octahydropyrido[2′,3′:3,4]pyrrolo[1,2-a]indol-5-one,
(4aR,11aR,11bS)-1-methyl-1,2,3,4,4a,11,11a,11b-octahydropyrido[2′,3′:3,4]pyrrolo-[1,2-a]indol-5-one,
   their enantiomers and diastereoisomers, and also addition salts thereof with a pharmaceutically acceptable acid or base.

It is understood that:

aryl means a phenyl or naphthyl group, each optionally being substituted by one or more halogen atoms, nitro, amino, linear or branched (C₁-C₆)alkyl or linear or branched (C₁-C₆)alkoxy groups,
an amino acid radical means the radicals alanyl, arginyl, asparaginyl, α-aspartyl, cysteinyl, α-glutamyl, glutaminyl, glycyl, histidyl, isoleucyl, leucyl, lysyl, methionyl, phenylalanyl, prolyl, seryl, threonyl, tryptophyl, tyrosyl and valyl,
arylalkyl means an aryl-alkyl group in which the alkyl group denotes a linear or branched chain of 1 to 6 carbon atoms and the aryl group denotes an optionally substituted phenyl or naphthyl group,
as an optionally substituted, 4- to 8-membered heterocycle optionally containing one or more double bonds within the heterocycle and optionally containing within the cyclic system a second hetero atom selected from an oxygen atom and a nitrogen atom, there may be mentioned, without implying any limitation, pyrrolidine, piperidine, azepane, piperazine and morpholine, those heterocycles optionally being substituted (including on the second nitrogen atom of piperazine) by one or more identical or different groups selected from linear or branched (C₁-C₆)alkyl, linear or branched (C₁-C₆)hydroxyalkyl, linear or branched (C₁-C₆)alkoxy-(C₁-C₆)alkyl, CO₂Rᵥ, CO₂—R_{w}—NRᵥR′ᵥ, CO₂—R_{w}—ORᵥ (wherein Rᵥ represents a hydrogen atom or a linear or branched (C₁-C₆)alkyl group, R′ᵥ is as defined for Rᵥ, and R_{w} represents a linear or branched (C₁-C₆)alkylene chain), aryl, aryloxycarbonyl, linear or branched aryl-(C₁-C₆)alkoxy-carbonyl, optionally substituted cycloalkyl, optionally substituted cycloalkylalkyl, optionally substituted heterocycloalkyl, optionally substituted heterocycloalkylalkyl, and aminoalkyl in which the alkyl moiety is a linear or branched chain of 1 to 6 carbon atoms and the amino moiety optionally is substituted by one or two identical or different, linear or branched (C₁-C₆)alkyl groups,
cycloalkyl means a saturated, 4- to 8-membered, monocyclic group,
cycloalkylalkyl means a cycloalkyl-alkyl group wherein the alkyl group denotes a linear or branched chain of 1 to 6 carbon atoms and the cycloalkyl group denotes a saturated, 4- to 8-membered, monocyclic group,
heterocycloalkyl means a saturated, 4- to 8-membered, monocyclic group containing 1 or 2 hetero atoms selected from nitrogen, oxygen and sulphur,
heterocycloalkylalkyl means a heterocycloalkyl-alkyl group wherein the alkyl group denotes a linear or branched chain of 1 to 6 carbon atoms and the heterocycloalkyl group denotes a saturated, 4- to 8-membered, monocyclic group containing 1 or 2 hetero atoms selected from nitrogen, oxygen and sulphur,
the expression “optionally substituted” when referring to the groups cycloalkyl, cycloalkylalkyl, heterocycloalkyl and heterocycloalkylalkyl means that those groups may be substituted by one or more identical or different substituents selected from linear or branched (C₁-C₆)alkyl, linear or branched (C₁-C₆)hydroxyalkyl, linear or branched (C₁-C₆)alkoxy-(C₁-C₆)alkyl, carboxy, linear or branched (C₁-C₆)alkoxy-carbonyl and aminoalkyl in which the alkyl moiety is a linear or branched chain of 1 to 6 carbon atoms and the amino moiety optionally is substituted by one or two identical or different, linear or branched (C₁-C₆)alkyl groups,
the expression “optionally substituted”, when referring to linear or branched (C₁-C₆)alkyl or linear or branched (C₂-C₆)alkenyl or arylalkyl groups means that these groups may be substituted by one or more halogen atoms or by one or more hydroxy groups, linear or branched (C₁-C₆)alkoxy groups or amino groups optionally substituted by one or two linear or branched, identical or different (C₁-C₆)alkyl groups,
a heterocycle having 5, 6 or 7 ring members means a saturated or unsaturated monocyclic group having 5, 6 or 7 sides and containing one or two hetero atoms selected from nitrogen and oxygen; pyrrolidine, piperidine, azepane and pyridine may be mentioned;
α, β, γ and δ mean the chiral centres that may be present in the compounds of formulae (III) and (IV),
the notation (5aRS,12aSR,12bSR,12cSR)- followed by the name of the compound means that the product obtained is a racemic mixture and that therefore both configurations are possible;
by way of example:
(5aRS,12aSR,12bSR,12cSR)-7-chloro-2,3,4,5,5a,10,11,12a,12b,12c-decahydro-1H,12H-3a,9b,11-triazabenzo[a]naphtho[2,1,8-cde]azulen-12-one means that the product obtained, the racemic mixture, contains (5aR,12aS,12bS,12cS)-7-chloro-2,3,4,5,5a,10,11,12a,12b,12c-decahydro-1H,12H-3a,9b,11-triazabenzo[a]naphtho[2,1,8-cde]azulen-12-one and (5aS,12aR,12bR,12cR)-7-chloro-2,3,4,5,5a,10,11,12a,12b,12c-decahydro-1H,12H-3a,9b,11-triazabenzo[a]naphtho[2,1,8-cde]azulen-12-one;
the notation (5aR,12aS,12bS,12cS)- or (5aS,12aR,12bR,12cR)- followed by the name of the compound means that the product obtained is an optically pure enantiomer;
by way of example:
(5aS,12aR,12bR,12cR)— or (5aR,12aS,12bS,12cS)-7-chloro-2,3,4,5,5a,10,11,12a,12b,12c-decahydro-1H,12H-3a,9b,11-triazabenzo[a]naphtho[2,1,8-cde]azulen-12-one means that the product obtained, the optically pure enantiomer, is (5aS,12aR,12bR,12cR)-7-chloro-2,3,4,5,5a,10,11,12a,12b,12c-decahydro-1H,12H-3a,9b,11-triazabenzo[a]naphtho[2,1,8-cde]azulen-12-one or (5aR,12aS,12bS,12cS)-7-chloro-2,3,4,5,5a,10,11,12a,12b,12c-decahydro-1H,12H-3a,9b,11-triazabenzo[a]naphtho[2,1,8-cde]azulen-12-one; enantiomer α and enantiomer β mean the optically pure enantiomers of the corresponding racemic mixture; by way of example:
(5aR,12aS,12bS,12cS)- or (5aS,12aR,12bR,12cR)-7-chloro-2,3,4,5,5a,10,11 ,12a,12b,12c-decahydro-1H,12H-3a,9b, 11-triazabenzo[a]naphtho[2, 1 ,8-cde]azulen-12-one (enantiomer α) means that if enantiomer α represents (5aR,12 aS,12bS,12cS)-7-chloro-2,3,4,5,5a,10,11,12a,12b,12c-decahydro-1H,12H-3a,9b,11-triazabenzo[a]naphtho[2,1,8-cde]azulen-12-one then enantiomer β represents (5aS,12aR,12bR,12cR)-7-chloro-2,3,4,5,5a,10,11,12a,12b,12c-decahydro-1H,12H-3a,9b,11-triazabenzo[a]naphtho[2,1,8-cde]azulen-12-one.

Other neuroprotective compounds according to the invention are, more especially, compounds of formula (V):

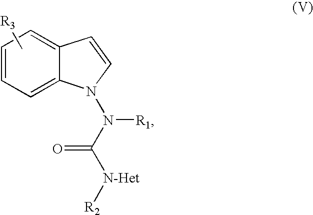

wherein:
R₁ and R₂, which may be the same or different, represent a hydrogen atom or a linear or branched (C₁-C₆)alkyl group,
R₃ represents a-hydrogen or halogen atom, a linear or branched (C₁-C₆)alkyl group, or a linear or branched (C₁-C₆)alkoxy group,
Het represents a pyridyl, pyrimidinyl or piperidyl group, optionally substituted by one or more groups selected from halogen, linear or branched (C₁-C₆)alkyl and linear or branched (C₁-C₆)alkoxy,
[symbol] represents a single bond or a double bond,
   it being understood that R₃ may be attached to any of the carbons of the indole/indoline nucleus that allows it,
   their enantiomers and diastereoisomers, and also addition salts thereof with a pharmaceutically acceptable acid or base.

Even more preferably, the neuroprotective compounds of formula (V) according to the invention are:
N-(1H-indol-1-yl)-N′-(3-pyridyl)urea,
N-(2,3-dihydro-1H-indol-1-yl)-N′-(3-pyridyl)urea.

The present invention relates also to pharmaceutical compositions comprising, as active ingredient, at least one neuroprotective compound, for example the compounds of formulae (I), (II), (III), (IV) and (V), its enantiomers, diastereoisomers or one of its addition salts with a pharmaceutically acceptable acid or base, alone or in combination with one or more pharmaceutically acceptable, inert, non-toxic excipients or carriers.

The pharmaceutical compositions thereby obtained will generally be presented in a dosage form; for example, they may take the form of tablets, dragées, capsules, suppositories, or injectable or drinkable solutions and may be administered by the oral, rectal, intramuscular or parenteral route.

Among the pharmaceutical compositions according to the invention there may be mentioned more especially those that are suitable for oral, parenteral (intravenous, intramuscular or subcutaneous), per- or trans-cutaneous, intravaginal, rectal, nasal, perlingual, buccal, ocular or respiratory administration.

The pharmaceutical compositions according to the invention for parenteral injections especially include aqueous and non-aqueous sterile solutions, dispersions, suspensions or emulsions as well as sterile powders for the reconstitution of injectable solutions or dispersions.

The pharmaceutical compositions according to the invention for solid oral administration especially include tablets or dragées, sublingual tablets, sachets, capsules and granules, and for liquid oral, nasal, buccal or ocular administration especially include emulsions, solutions, suspensions, drops, syrups and aerosols.

The pharmaceutical compositions for rectal or vaginal administration are preferably suppositories or ovules, and those for per- or trans-cutaneous administration especially include powders, aerosols, creams, ointments, gels and patches.

The above-mentioned pharmaceutical compositions illustrate the invention but do not limit it in any way.

Among the pharmaceutically acceptable, inert, non-toxic excipients or carriers there may be mentioned, by way of example and without implying any limitation, diluents, solvents, preservatives, wetting agents, emulsifiers, dispersants, binders, swelling agents, disintegrants, retardants, lubricants, absorbency agents, suspension agents, colourants, flavourings etc.

The useful dosage varies according to the age and weight of the patient, the route of administration, the pharmaceutical composition used, the nature and severity of the disorder, and the administration of any associated treatments. The dosage ranges from 0.1 mg to 100 mg per day in one or more administrations.

The following Examples illustrate the invention but do not limit it in any way.

The starting materials used are known products or are prepared according to known procedures. The various Preparations yield synthesis intermediates that are useful in preparation of compounds of the invention.

The structures of the compounds described in the Examples and in the Preparations were determined in accordance with the usual spectrometric techniques (infrared, nuclear magnetic resonance, mass spectrometry etc.).

The melting points were determined using a TOTTOLI apparatus (without emergent column correction). When the compound is in the form of a salt, the melting point corresponds to that of the compound in salt form.

### PREPARATION 1

### O-diphenylphosphinylhydroxylamine

To an aqueous solution of 149.44 g of hydroxylamine hydrochloride (340 ml of water) there are added a solution of 72.75 g of sodium hydroxide (290 ml of water-) and 960 ml of 1,4-dioxane. The mixture is cooled to −15° C. and then, after 15 minutes, a solution of 180 g of diphenylphosphinyl chloride in 725 ml of dioxane is added all at once with mechanical stirring.

After 5 minutes, 3 litres of water are added all at once. A white precipitate forms which is filtered off and then taken up in 0.25M sodium hydroxide solution at 0° C. The mixture is mechanically stirred at 0° C. for 30 minutes before being filtered again. The precipitate is dried in vacuo (phosphorus pentoxide) to yield 72.5 g of the expected product.

Melting point: 104° C.
Elemental microanalyses:

| | C % | H % | N % |
|---|---|---|---|
| Calculated: | 61.80 | 5.19 | 6.01 |
| Found: | 61.20 | 5.07 | 5.72 |

### PREPARATION 2

### N-aminoindole

To a suspension of 177.72 g of ground potassium hydroxide in 1.3 litres of DMF there are added 21.85 g of indole and then, all at once, 72.5 g of a suspension of the compound of Preparation 1 in 1.3 litres of DMF. The thick mixture is heated at between 60 and 70° C. for 3 hours 30 minutes with mechanical stirring and then, whilst hot, is poured into 3.5 litres of ice-cold water. After cooling, the resulting solution is extracted 3 times using 1.5 litres of ethyl ether. The organic phase is dried over sodium sulphate, filtered and then concentrated under reduced pressure. Chromatography on silica gel (cyclohexane/ether: 80/20 and then 50/50) allows 16.5 g of the expected product to be obtained.

Melting point: 35° C.
Elemental microanaylses:

| | C % | H % | N % |
|---|---|---|---|
| Calculated: | 72.70 | 6.10 | 21.20 |
| Found: | 72.68 | 6.14 | 21.17 |

### PREPARATION 3

### 5-Chloro-N-aminoindole

The product is obtained according to the procedure of Preparation 2, using 5-chloroindole instead of indole.

Melting point: 46° C.
Elemental microanalyses:

| | C % | H % | N % |
|---|---|---|---|
| Calculated: | 57.61 | 4.23 | 16.81 |
| Found: | 57.51 | 4.41 | 16.68 |

### PREPARATION 4

### Methyl 1-(2-chloroethyl)-1,2,5,6-tetrahydropyridine-3-carboxylate

### Step A: Guvacine Hydrochloride

7 g of arecoline hydrobromide are dissolved in 20 ml of water, the solution is made alkaline by adding 5.13 g of potassium carbonate and is then saturated with NaCl. The aqueous phase is extracted three times with diethyl ether. The combined organic phases are dried over sodium sulphate, filtered and then evaporated until a weight of 4.7 g of a colourless oil is obtained. The oil is taken up in 20 ml of toluene; the solution is turbid. After adding sodium sulphate and filtering, the insoluble material is washed with 13 ml of toluene. 3.92 ml of 1-chloroethyl chloroformate are added to the organic solution. A precipitate forms and the reaction mixture is heated for 12 hours at the reflux of toluene. The precipitate is filtered off and then the organic phase is washed with 0.1M aqueous hydrochloric acid solution; the aqueous phase is extracted once with diethyl ether. The combined organic phases are dried over sodium sulphate, filtered and then evaporated under reduced pressure. The residue is taken up in 25 ml of methanol and heated at reflux for 2 hours. The methanol is evaporated off under reduced pressure, and 3.8 g of the expected product are obtained in a yield of 73%. Guvacine base is obtained by dissolving the hydrochloride in water; the aqueous phase is made alkaline by adding potassium carbonate until a pH of 10 is achieved, and it is saturated with NaCl. The aqueous phase is extracted three times with diethyl ether and the combined organic phases are dried over sodium sulphate, filtered and then evaporated until 3 g of a colourless oil are obtained.

### Elemental Microanalyses:

| | C % | H % | N % |
|---|---|---|---|
| Calculated: | 47.33 | 6.81 | 7.89 |
| Found: | 47.38 | 6.93 | 7.83 |

### Step B: Methyl 1-(2-chloroethyl)-1,2,5,6-tetrahydropyridine-3-carboxylate

To a suspension of 530 mg of the compound of Step A above in 12 ml of acetone there are added 2.53 ml of triethylamine and 1.1 ml of 1-bromo-2-chloroethane. The mixture is stirred at ambient temperature for 18 hours and then heated at reflux for 8 hours. It is evaporated under reduced pressure, taken up in dichloromethane and washed with aqueous potassium carbonate solution. The aqueous phase is extracted with dichloromethane. The combined organic phases are dried over sodium sulphate, filtered and then evaporated under reduced pressure. The residue is purified by flash chromatography over silica gel (cyclohexane/AcOEt:7/3) allowing 430 mg of the expected compound to be obtained.

### Elemental Microanalyses:

| | C % | H % | N % |
|---|---|---|---|
| Calculated: | 53.08 | 6.93 | 6.88 |
| Found: | 53.74 | 7.22 | 6.72 |

INFRARED (ν_{cm-1}):

2951; 2917; 2811 (νC—H); 2767 (νN—CH₂); 1709 (νC═O); 1657 (νC═C); 1462; 1436 (δC—H); 1375 (νC—N); 1261 (νC—O).

### PREPARATION 5

### Methyl 1-[2-(4-methylpiperazin-1-yl)ethyl]-1,2,5,6-tetrahydro-pyridine-3-carboxylate

To a solution of 320 mg of the compound of Preparation 4 in 5 ml of 70% aqueous ethanol there are added 540 μl of 1-methylpiperazine. The solution is stirred at ambient temperature for 72 hours and is then evaporated under reduced pressure. The residue is taken up in dichloromethane and washed twice with aqueous sodium carbonate solution. The organic phase is dried over sodium sulphate, filtered and evaporated under reduced pressure to obtain 320 mg of the expected compound.

INFRARED (ν_{cm-1}):

2938 (νC—H); 2793 (νN—CH₃); 1712 (νC═O); 1657 (νC═C); 1438 (δC—H); 1373 (νC—N); 1262 (νC—O).

### PREPARATION 6

### 1,2-Bis[3-(ethoxycarbonyl)-1,2,5,6-tetrahydropyridin-1-yl]-ethane

To a solution of 900 mg of the compound of Step A of Preparation 4 in 10 ml of methanol there is added 0.32 ml of 2-bromochloroethane, followed by 1.6 ml of triethylamine. The mixture is heated at reflux for 20 hours and is evaporated under reduced pressure. The residue is dissolved in dichloromethane and extracted with saturated aqueous potassium carbonate solution. The aqueous phase is extracted with dichloromethane. The combined organic phases are dried over sodium sulphate, filtered and evaporated under reduced pressure. Flash chromatography over silica gel (AcOEt, then AcOEt/MeOH:95/5 to 90/10) allows 500 mg of the expected product to be obtained.

Melting point: 77° C.

### Elemental Microanalyses:

| | C % | H % | N % |
|---|---|---|---|
| Calculated: | 61.13 | 7.91 | 8.91 |
| Found: | 61.17 | 7.76 | 8.80 |

INFRARED (ν_{cm-1}):

2950; 2908 (νC—H); 2807 (νN—CH₂); 1708 (νC═O); 1656 (νC═C); 1435 (δC—H); 1351 (νC—N); 1258 (νC—O).

### PREPARATION 7

### tert-Butyl (RS)-2-[(2SR,3SR)-3-(methoxycarbonyl)-1-allylpiperidin-2-yl]indoline-1-carboxylate

### Step A: 1-(tert-Butoxycarbonyl)-1H-indol-2-yl-2-boronic acid

75 ml of a 2M solution of LDA is added dropwise, at 0° C., to a solution of 25 g of tert-butyl 1H-indole-1-carboxylate and 32.4 g of triisopropyl borate in 120 ml of anhydrous THF under a nitrogen atmosphere. Stirring is maintained for 40 min. before the addition of 200 ml of aqueous 2M hydrochloric acid solution. The pH is adjusted to pH=7 by adding a concentrated solution of ammonium hydroxide. After separation of the phases, the aqueous phase is extracted with ethyl acetate (2×100 ml). The organic phases are collected, washed with saturated sodium chloride solution (100 ml), dried over sodium sulphate, filtered and concentrated under reduced pressure. Water is added to the residue and the mixture is triturated until precipitation of the boronic acid occurs, which is filtered off and washed four times with pentane to obtain 29.27 g of the expected product.

Melting point: 96-98° C.

### Step B: tert-Butyl 2-[(3-methoxycarbonyl)pyridin-2-yl]indole-1-carboxylate

13.46 g of a solution of sodium carbonate in 50 ml of water and 4.77 g of tetrakis(triphenylphosphine)palladium are added in succession to a degassed solution of 9.94 g of methyl 2-bromonicotinate in 170 ml of DME at 85° C. A solution of 12.96 g of the compound of the above Step A in 50 ml of ethanol is then added dropwise. Stirring is maintained for 6 h at 85° C. before returning to ambient temperature and adding 200 ml of water. After separation of the phases, the aqueous phase is extracted with diethyl ether (2×100 ml). The organic phases are collected, washed with saturated sodium chloride solution, dried over sodium sulphate, filtered and concentrated under reduced pressure. Chromatography on a silica gel column (cyclohexane/ethyl acetate: 9/1) allows 14.11 g of the expected product to be obtained.

Melting point: 83° C.
Mass spectrometry (ES+, m/z): 375 (M+Na)⁺; 353 (M+H)⁺.

### Elemental Microanalyses:

| | C % | H % | N % |
|---|---|---|---|
| Calculated: | 68.17 | 5.72 | 7.95 |
| Found: | 68.03 | 5.85 | 7.85 |

### Step C: tert-Butyl (SR)-2-[(2RS,3SR)-3-(methoxycarbonyl)piperidin-2-yl]indoline-1-carboxylate

A mixture of 6.00 g of the compound of Step B above and 7.0 g of 5% rhodium on alumina in 60 ml of acetic acid is stirred at ambient temperature under 15 bars of hydrogen pressure a for 20 h. The reaction mixture is then filtered through paper. The paper is then rinsed with methanol. The filtrate is concentrated under reduced pressure and the residue is taken up in dichloromethane and water. Potassium carbonate is added until the pH of the aqueous phase is basic. The phases are separated and the aqueous phase is extracted twice with dichloromethane. The organic phases are combined, dried over sodium sulphate, filtered and concentrated under reduced pressure. Chromatography on a silica gel column (dichloromethane/methanol: 95/5) allows 3.49 g of the expected compound to be obtained.

Melting point: 79-80° C.
Mass spectrometer (ES+, m/z): 383 (M+Na)⁺; 361 (M+H)⁺; 305.

### Elemental Microanalyses:

| | C % | H % | N % |
|---|---|---|---|
| Calculated: | 66.64 | 7.83 | 7.77 |
| Found: | 66.45 | 7.96 | 7.67 |

### Step D: tert-Butyl (SR)-2-[(2RS,3SR)-3-(methoxycarbonyl)-1-allylpiperidin-2-yl]indoline-1-carboxylate

0.3 ml of allyl bromide and then 800 mg of potassium carbonate are added in succession at ambient temperature to a solution of 440 mg of the compound of Step C above 2 in 10ml of acetonitrile. The mixture is stirred at ambient temperature for 3 h and then water is added. The aqueous phase is extracted with dichloromethane. The combined organic phases are dried over sodium sulphate, filtered and concentrated under reduced pressure. Chromatography on a silica gel column (cyclohexane/ethyl acetate: 8/2) allows 382 mg of the expected product to be isolated.

Melting point: 119-121° C.

### Elemental Microanalyses:

| | C % | H % | N % |
|---|---|---|---|
| Calculated: | 68.97 | 8.05 | 6.99 |
| Found: | 68.85 | 8.15 | 7.04 |

### Step E: tert-Butyl (RS)-2-[(2SR,3SR)-3-(methoxycarbonyl)-1-allylpiperidin-2-yl]indoline-1-carboxylate

0.80 g of a solution of the compound of Step D above in 10 ml of THF is added to a solution of sodium methanolate in methanol (prepared by adding 0.23 g of sodium in small portions to 10 ml of methanol at 0° C.). The reaction mixture is heated for 3 hours with reflux of the solvent. After the reaction mixture has been cooled to ambient temperature, 15 ml of water are added; the methanol and THF are removed by evaporation under reduced pressure. The resulting solution is extracted twice with ethyl acetate. The organic phases are combined, dried over sodium sulphate, filtered and concentrated under reduced pressure. Chromatography on a silica gel column (cyclohexane/diethyl ether: 9/1) allows 0.58 g of the expected product to be isolated.

Infrared (ν_{cm-1}:

2929 (ν_{C—H);} 1732 (ν_{C═O} ester); 1698 (ν_{C═O} carbamate); 1483 (ν_{C═C}); 1369 (δ_{C—H} tBu).

### PREPARATION 8

### Methyl ((2RS,3SR)-2-[(SR)-1-nitrosoindolin-2-yl]piperidine-3-carboxylate

### Step A: Methyl (2RS,3SR) 2-[(SR)-indolin-2-yl]piperidine-3-carboxylate

15 ml of trifluoroacetic acid are added at ambient temperature to a solution of 927 mg of the compound of Step C of Preparation 7 in 15 ml of dichloromethane. The mixture is stirred at ambient temperature for 2 h and concentrated under reduced pressure. The residue is taken up in dichloromethane and water. Potassium carbonate is added until the pH of the aqueous phase is basic. The aqueous phase is extracted with dichloromethane. The organic phases are combined, dried over sodium sulphate and concentrated under reduced pressure to yield 633 mg of the expected product.

Infrared (ν_{cm-1})

3369 (ν_{N—H}); 2942 and 2857 (ν_{C—H}); 1720 (ν_{C═O}); 1485 (ν_{C═C);} 1249, 1197 and 1165 (ν_{N—C}).

### Step B: Methyl (2RS,3SR)-2-[(SR)-1-nitrosoindolin-2-yl]piperidine-3-carboxylate

633 mg of the compound of the above Step A are-dissolved in a mixture of 6 ml of acetic acid and 6 ml of water at 0° C. and a solution of 168 mg of sodium nitrite in 4 ml of water is added dropwise. The mixture is stirred for 20 min. at 0° C. before adding dichloromethane. Potassium carbonate is added until the pH of the aqueous phase is basic. The aqueous phase is extracted twice with dichloromethane. The organic phases are collected, dried over sodium sulphate and concentrated under reduced pressure. Chromatography on a silica gel column (dichloromethane/methanol: 99/1) allows 538 mg of the expected product to be isolated.

Infrared (ν_{cm-1}:

3314 (ν_{N—H}); 2939 and 2857 (ν_{C—H}); 1721 (ν_{C═O}); 1485 and 1477 (ν_{C═C}); 1430 (ν_{N═O}); 1168 (ν_{C—N}).

### PREPARATION 9

### Ethyl (1SR,7aRS,12aSR,12bSR)-9-chloro-1,2,3,4,6,7,7a,12,12a,12b-decahydroindolo[2,3-α]guinolizine-1-carboxylate

### Step A: 1-[2-(5-Chloro-1H-indol-3-yl)ethyl]piperidin-2-one

To a solution of 100 g of 5-chlorotryptamine hydrochloride in 1.4 litres of 2-methoxyethanol there are added 60 g of Na₂CO₃. The reaction mixture is stirred at reflux under nitrogen. A solution of 111.2 g of 5-bromovalerate in 200 ml of 2-methoxyethanol is added dropwise over a period of 5-6 hours and the mixture is heated at reflux for 24 hours. After cooling, the reaction mixture is filtered over Celite and the filtrates are concentrated under reduced pressure. The oil is extracted with 500 ml of CH₂Cl₂ and 3000 ml of water. The organic phases are washed with saturated sodium chloride solution, then dried over Na₂SO₄ and concentrated under reduced pressure. The solid is recrystallised from a 9/1 acetone/pentane mixture to yield 107 g of the expected product.

Melting point. 155° C.
Mass spectrometry (EI, m/z): 276.8 (M⁺).

### Step B: 9-Chloro-2,3,4,6,7,12-hexahydro-1H-indolo-[2,3-a]quinolizin-5-ylium tetrafluoroborate

To a solution of the compound of Step A above in 1.2 litres of toluene there are added 83 ml of POCl₃. The reaction mixture is heated at 90° C. for 5 hours under nitrogen. After cooling whilst stirring the mixture, 3 to 4 litres of water are added and then allowed to separate. 436 ml of a solution of HBF₄ are added dropwise to the aqueous phase. 98 g of the expected product are obtained after filtration, washing with water and then drying over P₂O₅ under.

Melting point: >280° C.
Mass spectrometry(EI, m/z): 346.5 (M⁺).

### Step C: 9-Chloro-2,3,4,6,7,12-hexahydroindolo[2,3-a]quinolizine

61 g of the product of Step B above are dissolved in 510 ml of methanol and 115 ml of deionised water. The reaction mixture is stirred vigorously and heated at reflux for 2.5 hours until dissolution occurs. Refluxing is stopped and 115 ml of 4M NaOH solution are added dropwise. After the addition is complete, the reaction mixture is cooled to 0-5° C., and 35 ml of 4M NaOH solution are added with vigorous stirring for 0.5 hour. The solid residue is filtered off, washed with water and dried over P₂O₅ in vacuo to yield 42 g of the expected product.

Melting point: 114° C.
Mass spectrometry (EI, m/z): 257.78 (M⁺).

### Step D: Ethyl (IRS)-9-chloro-2,3,4,6,7,12-hexahydroindolo[2,3-a]quinolizine-1-carboxylate

To a solution of 25 g of the compound of Step C above in 500 ml of distilled CH₂Cl₂ there are added 1.75 g of 4-(dimethylamino)pyridine and 25 ml of DIEA under an argon atmosphere and the batch is stirred at ambient temperature until dissolution occurs. A solution of 19 ml of ClCO₂Et (97%) in distilled dichloromethane is added. After stirring at ambient temperature for 12 hours, the reaction mixture is filtered, the insoluble material is rinsed with 120 ml of CH₂Cl₂ and the filtrate is concentrated under reduced pressure. Chromatography over silica gel (CH₂Cl₂) followed by recrystallisation from a 9/1 acetone/pentane mixture makes it possible to obtain 22 g of the expected product.

Melting point: 128-130° C.
Mass spectrometry (EI m/z): 330.82 (M⁺).

### Step E: Ethyl (1SR,12bRS)-9-chloro-1,2,3,4,6,7,12,12b-octahydroindolo[2,3-a]quinolizine-1-carboxylate (cis diastereoisomer)

16 ml of acetic acid are added to a solution of 16 g of the product of Step D above in 300 ml of distilled THF. 4.4 g of NaBH₃CN are added in small portions under a nitrogen atmosphere and at 0° C.; the reaction mixture is then stirred vigorously at ambient temperature for 12 hours. Saturated Na₂CO₃ solution is then added at 0° C.; the solvent is then evaporated off under reduced pressure. 200 ml of CH₂Cl₂ and 80 ml of water are added to the residue. After extraction with CH₂Cl₂, the organic phases are washed with saturated sodium chloride solution, dried over Na₂SO₄ and then concentrated under reduced pressure. 20 ml of 2M HCl solution are added to the crude reaction mixture in 350 ml of ethanol and the reaction mixture is heated at reflux for 5 hours. The reaction mixture is concentrated under reduced pressure and the solid is dissolved in 270 ml of CH₂Cl₂. This organic solution is added to 130 ml of water. The solution is made alkaline (pH=8-9) by adding saturated Na₂CO₃ solution and is extracted with CH₂Cl₂. The organic phases are combined, washed with saturated sodium chloride solution, dried over Na₂SO₄ and then concentrated under reduced pressure. Recrystallisation from a 9/1 acetone/pentane mixture makes it possible to obtain 15 g of the expected product.

Melting point: 184° C.

### Elemental Microanalyses:

| | C % | H % | N % |
|---|---|---|---|
| Calculated: | 64.95 | 6.36 | 8.41 |
| Found: | 65.01 | 6.39 | 8.36 |

### Step F: Ethyl (1SR,12bSR)-9-chloro-1,2,3,4,6,7,12,12b-octahydroindolo[2,3-a]quinolizine-1-carboxylate (trans diastereoisomer)

Under a flow of argon and at 0° C., 2.5 g of NaH are added in small portions to a solution of a 9 g of the compound of Step E above in 300 ml of DME. After stirring at ambient temperature for 12 hours, the reaction mixture is cautiously poured onto ice and stirred for 1 hour. The organic solvent is evaporated off and the aqueous phase is cooled to 0-5° C., at which temperature 4M HCl solution is added until the pH=2-4. After stirring, saturated Na₂CO₃ solution is added until the pH=9. The reaction mixture is extracted with AcOEt and the organic phases are dried over Na₂SO₄, filtered and then concentrated under reduced pressure. The crude reaction mixture is recrystallised from a minimum of AcOEt and the solid is dried in vacuo to yield 8.3 g of the expected product.

Melting point: 88-90° C.
Infrared (ν_{cm-1):}
3442; 2933; 1709

### Step G: Ethyl (1SR,7aRS,12aSR,12bSR)-9-chloro-1,2,3,4,6,7,7a,12,12a,12b-decahydroindolo[2,3-a]-quinolizine-1-carboxylate (trans diastereoisomer)

To a solution of 350 ml of TFA at 0° C. under a large flow of argon there are added, alternately and in small portions, 10.3 g of the product of Step F above and 15 g of NaBH₃CN. The reaction mixture is stirred for 10 minutes at ambient temperature between each addition. After 8 hours, 3 g of NaBH₃CK are added again and then stirred for a further 12 hours at ambient temperature. 20 ml of water are added dropwise to the reaction mixture at 0° C. followed by CH₂Cl₂ until dissolution occurs. After stirring at ambient temperature, the solvents (TFA, CH₂Cl₂) are evaporated off. The aqueous phase is cooled to 0° C. and 4M NaOH solution is added. After extracting with Et₂O, the organic phases are dried over Na₂SO₄, filtered and then concentrated under reduced pressure. Recrystallisation from Et₂O followed by chromatography on silica gel (CH₂Cl₂/MeOH: 100/5) makes it possible to obtain 8 g of the expected product.

Melting point: 126-129° C.
Mass spectrometry (EI, m/z): 335.2 [M+H]⁺.
Infrared (ν_{cm-1}):
3374; 2948, 1726

### PREPARATION 10

### Methyl 1-[2-(4-methylpiperazin-1-yl)ethyl]-1,2,5,6-tetrahydro-pyridine-3-carboxylate

To a solution of 320 mg of the compound of Preparation 4 in 5 ml of 70% aqueous ethanol there are added 540 μl of 1-methylpiperazine. The solution is stirred at ambient temperature for 72 hours and is then evaporated under reduced pressure. The residue is taken up in dichloromethane and washed twice with aqueous sodium carbonate solution. The organic phase is dried over sodium sulphate, filtered and evaporated under reduced pressure to obtain 320 mg of the expected compound.

Infrared (ν_{cm-1}):

2938 (νC—H); 2793 (νN—CH₃); 1712 (νC═O); 1657 (νC═C); 1438 (δC—H);

1373 (νC—N); 1262 (νC—O).

### PREPARATION 11

### Methyl 1-[2-(piperidin-1-yl)ethyl]-1,2,5,6-tetrahydropyridine-3-carboxylate

The product is obtained according to the procedure of Preparation 10 using piperidine instead of 1-methylpiperazine.

INFRARED (ν_{cm-1}):

2955; 2932; 2872 (νC—H); 1736 (νC═O); 1661 (νC═C); 1434 (δC—H); 1368;

1341 (νC—N); 1236; 1194 (νC—O).

### PREPARATION 12

### tert-Butyl (RS)-2-[(2SR,3SR)-3-(methoxycarbonyl)-1-allyl-piperidin-2-yl]indoline-1-carboxylate

### Step A: tert-Butyl (SR)-2-[(2RS,3SR)-3-(methoxycarbonyl)-1-allylpiperidin-2-yl]indoline-1-carboxylate

0.3 ml of allyl bromide and then 800 mg of potassium carbonate are added in succession at ambient temperature to a solution of 440 mg of the compound of Step C of Preparation 7 in 10 ml of acetonitrile. The mixture is stirred at ambient temperature for 3 h and then water is added. The aqueous phase is extracted with dichloromethane. The combined organic phases are dried over sodium sulphate, filtered and concentrated under reduced pressure. Chromatography on a silica gel column (cyclohexane/ethyl acetate: 8/2) allows 382 mg of the expected product to be isolated.

Melting point: 119-121° C.

### Elemental Micronanylses:

| | C % | H % | N % |
|---|---|---|---|
| Calculated: | 68.97 | 8.05 | 6.99 |
| Found: | 68.85 | 8.15 | 7.04 |

### Step B: tert-Butyl (RS)-2-[(2SR,3SR)-3-(methoxycarbonyl)-1-allylpiperidin-2-yl]indoline-1-carboxylate

0.80 g of a solution of the compound of Step A in 10 ml of THF is added to a solution of sodium methanolate in methanol (prepared by adding 0.23 g of sodium in small portions to 10 ml of methanol at 0° C.). The reaction mixture is heated for 3 hours with reflux of the solvent. After the reaction mixture has been cooled to ambient temperature, 15 ml of water are added; the methanol and THF are removed by evaporation under reduced pressure. The resulting solution is extracted twice with ethyl acetate. The organic phases are combined, dried over sodium sulphate, filtered and concentrated under reduced pressure. Chromatography on a silica gel column (cyclohexane/diethyl ether: 9/1) allows 0.58 g of the expected product to be isolated.

Infrared (ν_{cm-1}): 2929 (ν_{C—H}); 1732 (ν_{C═O} ester); 1698 (ν_{C═O} carbamate); 1483 (ν_{C═C}); 1369 (δ_{C—H} tBu).

### PREPARATION 13

### Nicotinoyl azide

To 2.4 ml of concentrated hydrochloric acid (37%) there are added, at 0° C., 2 g of nicotinoylhydrazide and then a solution of 2.02 g of sodium nitrite in 3.6 ml of water. The reaction mixture is stirred at 0° C. for 30 minutes and then treated -with saturated sodium hydrogen carbonate solution. After extraction with diethyl ether (3 times), the organic phase is washed successively with water and with saturated sodium chloride solution before being dried over magnesium sulphate. After concentrating under reduced pressure, the expected product is obtained (G. Papeo et al., Synthesis, (2004), 2886).

Infrared(ν_{cm-1}): 2178 (νₙ₃); 1685 (ν_{co}).

### EXAMPLE 1

### (+) (2RS,7SR),(3RS,16RS)-10-Chloro-15-oxo-2,7,14,15-tetrahydro-14-aza-20,21-dinoreburnameninium chloride

The expected compound is obtained by chromatography, on a column of the Chiralpak AD type, of the compound of Example 1 described in the Patent Application EP 0 658 557.

### EXAMPLE 2

### N-(1H-indol-1-yl)-1-methyl-1,2,5,6-tetrahydropyridine-3-carboxamide

1.94 g of methyl 1-methyl-1,2,5,6-tetrahydropyridine-3-carboxylate hydrochloride are dissolved in 5 ml of water, and the solution is then made alkaline using potassium carbonate to achieve a pH of 10 and is then saturated with NaCl. The aqueous phase is extracted three times with diethyl ether. The combined organic phases are dried over Na₂SO₄, filtered and then evaporated. 1.3 g of the compound of Preparation 2 are dissolved in 26 ml of anhydrous dichloromethane and then, after cooling to −25° C., 9 ml of a 2M solution of trimethylaluminium in hexane are added. After 1 hour 30 minutes, a solution of 1.27 g of arecoline in 6.5 ml of anhydrous dichloromethane is added at ambient temperature. The reaction mixture is refluxed overnight and is then diluted with dichloromethane and poured into 50 ml of 20% aqueous sodium hydroxide solution. The organic phase is isolated and the aqueous phase is extracted with dichloromethane. The combined organic phases are washed with saturated NaCl solution, dried over sodium sulphate, filtered and then evaporated under reduced pressure. Flash chromatography on silica gel (CH₂Cl₂₁MeOH 95/5 and then 9/1) allows 470 mg of the expected product to be isolated.

Melting point: 194° C.

### Elemental Microanaylses:

| | C % | H % | N % |
|---|---|---|---|
| Calculated: | 70.56 | 6.71 | 16.46 |
| Found: | 70.35 | 6.80 | 16.36 |

Mass spectrometry (ESI+, m/z): 256.1 (M+H⁺); 278.1 (M+Na⁺).

### EXAMPLE 3

### N-(5-Chloroindol-1-yl)-1-[2-(4-methylpiperazin-1-yl)ethyl]-1,2,5,6-tetrahydropyridine-3-carboxamide

To a solution of 782 mg of the compound of Preparation 3 in 12 ml of anhydrous dichloromethane, previously cooled to −20° C., there are added 4.30 ml of a 2M solution of trimethylaluminium in hexane. The solution is stirred under nitrogen for 1 hour 30 minutes allowing the temperature to gradually come back up to ambient temperature. 1.05 g of a solution of the compound of Preparation 5 in 8 ml of anhydrous dichloromethane are then added. The mixture is heated at reflux under nitrogen for 16 hours. To the reaction mixture, cooled to 0° C., there are added 100 ml of 1M aqueous HCl solution (slow addition at the start), and then 250 ml of water. A first extraction using 3×100 ml of dichloromethane removes the excess of N-amino-5-chloroindole. The aqueous phase is made alkaline using saturated sodium carbonate solution until a pH of 10 is achieved, and it is extracted using 3×100 ml of dichloromethane. The organic phase is dried over sodium sulphate, filtered and concentrated under reduced pressure. Chromatography on a silica column (NH₄OH/MeOH/CH₂Cl₂: Jan. 1, 1998 to Feb. 18, 1980), followed by recrystallisation from 15 ml of a mixture of iPrOH/AcOEt (1/2), allows 470 mg of the expected product to be obtained.

Melting point: 157° C.

### Elemental Microanalyses:

| | C % | H % | N % |
|---|---|---|---|
| Calculated: | 62.75 | 7.02 | 17.42 |
| Found: | 62.50 | 6.92 | 17.23 |

Mass spectrometry (ESI+, m/z): 402 (M+1).
Infrared (ν_{cm-1}):

2946 to 2814 (ν CH); 1681 (ν CO); 1650; 1531; 1465.

### EXAMPLE 4

### N-(Indol-1-yl)-1-[2-[3-(ethoxycarbonyl)-1,2,5,6-tetrahydropyridin-1-yl]ethyl]-1,2,5,6-tetrahydropyridine-3-carboxamide

1 g of the compound of Preparation 2 is dissolved in 10 ml of anhydrous dichloromethane and then the reaction mixture is cooled to −20° C. 5.5 ml of a 2M solution of trimethyl-aluminium in hexane are added and the reaction mixture is stirred for 1 hour 30 minutes whilst allowing the temperature to increase. A solution of 1.5 g of the compound of Preparation 6 in 5 ml of dichloromethane is added and the reaction mixture is refluxed for 12 hours. The reaction mixture is diluted with dichloromethane and then poured into 20% aqueous sodium hydroxide solution. The organic phase is separated off and washed, first with 20% sodium hydroxide solution and then with saturated NaCl solution. The organic phase is dried over sodium sulphate, filtered and then evaporated under reduced pressure. Flash chromatography over silica gel (AcOEt, then AcOEt/CH₃OH:95/5) allows 180 mg of the expected product to be isolated.

Melting point: 82° C.

### Elemental Microanalyses:

| | C % | H % | N % |
|---|---|---|---|
| Calculated: | 67.63 | 6.91 | 13.72 |
| Found: | 67.41 | 7.09 | 13.63 |

Infrared (ν_{cm-1}):

3265 (νN—H); 2948; 2915; 2802 (νC—H); 1708; 1673 (νC═O); 1646; 1614;

1519 (νC═C); 1459; 1436 (δC—H); 1371; 1351 (νC—N); 1261; 1223; 1194 (νC—O).

### EXAMPLE 5

### (4aSR,11aRS,11bSR)-1-Allyl-1,2,3,4,4a,11,11a,11b-octahydropyrido[2′,3′:3,4]pyrrolo-[1,2-a]indol-5-one

Trifluoroacetic acid is added at ambient temperature to a solution of 80 mg of the compound of Preparation 7 in 3 ml of dichloromethane. The mixture is stirred at ambient temperature for 6 h. The reaction mixture is concentrated, under reduced pressure. The residue is taken up in dichloromethane and water. Potassium carbonate is added until the pH of the aqueous phase is basic. After separation of the phases, the aqueous phase is extracted with dichloromethane. The organic phases are collected, filtered and concentrated under reduced pressure. Chromatography on a silica gel column (cyclohexane/ethyl acetate: 1/1) allows 36 mg of the expected product to be isolated.

Melting point: 163° C.
Mass spectrometry (ES+, m/z): 291 (M+Na)⁺.

### Elemental Microanalyses:

| | C % | H % | N % |
|---|---|---|---|
| Calculated: | 76.09 | 7.51 | 10.44 |
| Found: | 76.00 | 7.61 | 10.37 |

### EXAMPLE 6

### (4aSR,11aSR,11bRS)-1,2,3,4,4a,11,11a,11b-Octahydro-1,6,6a-triaza-benzo[a]fluoren-5-one

1.14 g of zinc and 1.67 g of ammonium carbonate are added in succession to a solution of 0.56 g of the compound of Preparation 8 in 18 ml of ethanol and 9 ml of water at 0° C. The mixture is stirred at 0° C. for 20 min. and then filtered. The solid is washed with water and dichloromethane. The phases of the filtrate are separated. The aqueous phase is twice extracted with dichloromethane. The organic phases are combined, dried over sodium sulphate and concentrated under reduced pressure. The residue is purified by chromatography on a silica gel column (dichloromethane/methanol 9/1) to yield a mixture of compounds. That mixture is then dissolved in 10 ml of anhydrous dichloromethane and cooled to −20° C. under a nitrogen atmosphere. 1.2 ml of 2M trimethylaluminium solution is added to that solution. The reaction mixture is stirred at −20° C. for 90 min. and then refluxed for 16 h before being cooled and poured into 24 ml of aqueous 1M hydrochloric acid solution. The phases are separated. Potassium carbonate is added to the aqueous phase until a basic pH is obtained. That solution is then twice extracted with dichloromethane. The organic phase is dried over sodium sulphate, filtered and concentrated under reduced pressure. Chromatography on a silica. gel column (ethyl acetate/methanol 9/1) allows 158 mg of the expected product to be obtained.

Melting point: 211° C.
Mass spectrometry (ES+, m/z): 266 (M+Na)⁺; 244 (M+H)⁺.

### Elemental Microanalyses:

| | C % | H % | N % |
|---|---|---|---|
| Calculated: | 69.11 | 7.04 | 17.24 |
| Found: | 68.89 | 7.21 | 17.11 |

### EXAMPLE 7

### (5aS,12aR,12bR,12cR)- or (5aR,12aS,12bS,12cS)-2,3,5a,12a,12b,12c-Hexahydro-1H,4H-3a,9b,11-triazabenzo[a]naphtho[2,1,8-cde]azulene-10,12(5H,11H)-dione (enantiomer α)

### Step A: Ethyl (1SR,7aRS,12aSR,12bSR)-12-(aminocarbonyl)-9-chloro-1,2,3,4,6,7,7a,12,12a,12b-decahydroindolo[2,3-a]quinolizine-1-carboxylate

A solution of 385 mg of the compound of Preparation 9 in 6 ml of anhydrous THF is added, all at once and using a syringe, to a solution of 2.33 g of KOCN and 4.43 ml of trifluoroacetic acid in 12 ml of anhydrous THF. The reaction mixture is stirred at ambient temperature under nitrogen for 2 hours. After removing the solvent in vacuo, dichloromethane (30 ml) is added to the residue. The organic phase is extracted with HCl solution (1M, 6×10 ml). The aqueous phase is adjusted to pH 9 using Na₂CO₃ and is extracted with dichloromethane (3×20 ml). The organic phase is dried over Na₂SO₄, filtered and evaporated in vacuo to yield 500 mg of the expected product. The product is used immediately in the next Step.

### Step B: (5aRS,12aSR,12bSR,12cSR)-7-Chloro-2,3,5a,12a,12b,12c-hexahydro-1H,4H-3a,9b,11-triazabenzo[a]naphtho[2,1,8-cde]azulene-10,12(5H,11H)-dione

A solution of 500 mg of the product of Step A above in 25 ml of ethanol is treated with 6.36 mg of K₂CO₃. The reaction mixture is heated at reflux for 1 h. After removal of the solvent in vacuo, water (50 ml) is added and extraction with dichloromethane (3×20 ml) is carried out. The organic phase is dried over Na₂SO₄, filtered and evaporated in vacuo. Crystallisation from ethanol (50-ml) makes-it possible to obtain 220 mg-of the expected product.

Melting point: 243° C.
Mass spectrometry(EI, m/z): 331.

### Elemental Microanalyses:

| | C % | H % | N % |
|---|---|---|---|
| Calculated: | 61.54 | 5.47 | 12.66 |
| Found: | 61.39 | 5.54 | 12.58 |

### Step C (5aRS,12aSR,12bSR,12cSR)-2,3,5a,12a,12b,12c-Hexahydro-1H,4H-3a,9b,11-triazabenzo[a]naphtho[2,1,8-cde]azulene-10,12(5H,11H)-dione

To a solution of 109 mg of the compound of Step B above in 10 ml of anhydrous THF to which 140 μl of triethylamine has been added there is added a spatula “tip” of 10% palladium-on-carbon. The reactor is purged by means of several vacuum/nitrogen cycles and then the reaction mixture is placed under a hydrogen atmosphere and stirred at ambient temperature for 24 hours. The mixture is filtered over Celite and the solvent is removed by evaporation. HCl solution (1M, 30 ml) is added to the residue; the mixture is then extracted with dichloromethane (3×20 ml). The aqueous phase is brought to pH 9 using Na₂CO₃ and is extracted with dichloromethane (3×20 ml). The organic phase is dried over Na₂SO₄, filtered and evaporated in vacuo. Crystallisation from a minimum of ethanol makes it possible to obtain 65 mg of the expected product.

Melting point: 255° C.
Mass spectrometry (EI, m/z): 298.

### Elemental Microanalyses:

| | C % | H % | N % |
|---|---|---|---|
| Calculated: | 68.67 | 6.44 | 14.13 |
| Found: | 68.56 | 6.53 | 14.10 |

### Step D: (5aS,12aR,12bR,12cR)- or (5aR,12aS,12bS,12cS)-2,3,5a,12a,12b,12c-Hexahydro-1H,4H-3a,9b,11-triazabenzo[a]naphtho[2,1,8-cde]azulene-10,12(5H,11H)-dione (enantiomer α)

The compound of Step C above is resolved by fractional crystallisation of the diastereoisomeric salts prepared by adding to a methanolic solution either a solution of (−)-di-O,O′-para-toluoyl-L-tartaric acid or a solution of (+)-di-O,O′-para-toluoyl-D-tartaric acid. After separation of the diastereoisomeric salt, the base is isolated by customary treatment.

Melting point: 250° C.
Optical rotation α_{D}=+95° (c=1 in CHCl₃).

### Elemental Microanalyses:

| | C % | H % | N % |
|---|---|---|---|
| Calculated: | 68.15 | 6.48 | 14.03 |
| Found: | 68.12 | 6.62 | 14.01 |

### EXAMPLE 8

### (5aS,12aR,12bR,12cR)- or (5aR,12aS,12bS,12cS)-2,3,5a,12a,12b,12c-Hexahydro-1H,4H-3a,9b,11-triazabenzo[a]naphtho[2,1,8-cde]azulene-10,12(5H,11H)-dione (enantiomer ,β)

The compound of Step C of Example 7 is resolved by fractional crystallisation of the diastereoisomeric salts prepared by adding to a methanolic solution either a solution of (−)-di-O,O′-para-toluoyl-L-tartaric acid or a solution of (+)-di-O,O′-para-toluoyl-D-tartaric acid. After separation of the diastereoisomeric salt, the base is isolated by customary treatment.

Melting point: 250° C.
Optical rotation α_{D}=−95° (c=1 in CHCl₃).
Elemental microanalyses:

| | C % | H % | N % |
|---|---|---|---|
| Calculated: | 68.15 | 6.48 | 14.03 |
| Found: | 68.12 | 6.57 | 13.98 |

### EXAMPLE 9

### N-(Indol-1-yl)-1-[2-(4-methylpiperazin-1-yl)ethyl]-1,2,5,6-tetrahydropyridine-3-carboxamide

1 g of the compound of Preparation 10 is dissolved in 10 ml of anhydrous dichloromethane and then the reaction mixture is cooled to −20° C. 5.5 ml of a 2M solution of trimethyl-aluminium in hexane are added and the reaction mixture is stirred for 1 hour 30 minutes whilst allowing the temperature to increase. A solution of 1.5 g of the compound of Preparation 10 in 5 ml of dichloromethane is added and the reaction mixture is refluxed for 12 hours. The reaction mixture is diluted with dichloromethane and then poured into 20% aqueous sodium hydroxide solution. The organic phase is separated off and washed, first with 20% sodium hydroxide solution and then with saturated NaCl solution. The organic phase is dried over sodium sulphate, filtered and then evaporated under reduced pressure. Flash chromatography over silica gel (CH₂Cl₂/CH₃OH: 95/5, then 9/1 and 8/2) allows 380 mg of the expected product to be isolated.

Melting point: 130° C.

### Elemental Microanalyses:

| | C % | H % | N % |
|---|---|---|---|
| Calculated: | 68.63 | 7.95 | 19.06 |
| Found: | 68.49 | 8.09 | 18.93 |

Infrared (ν_{cm-1}):

3054 (ν═C—H); 2935 (νC—H); 2795 (νN—CH₃); 1681 (νC═O); 1646; 1614 (νC═C);

1521 (δN—H); 1458 (δC—H); 1372 (νC—N).

### EXAMPLE 10

### N-(Indol-1-yl)-1-(2-piperidin-1-yl-ethyl)-1,2,5,6-tetrahydropyridine-3-carboxamide

The product is obtained according to the procedure of Example 9 using the compound of Preparation 11 instead of the compound of Preparation 10.

Flash chromatography over silica gel (CH₂Cl₂/CH₃OH:9/1, then 8/2) allows 240 mg of the expected product to be isolated.

Melting point: 65° C.

### Elemental Microanalyses:

| | C % | H % | N % |
|---|---|---|---|
| Calculated: | 69.78 | 8.09 | 15.50 |
| Found: | 69.98 | 8.17 | 15.40 |

Infrared (ν_{cm-1}):

3238 (νN—H); 2931 (νC—H): 2788 (νN—CH₂); 1672 (vC═O); 1643 (vC═C);

1521 (δN—H); 1459 (δC—H); 1370 (νC—N).

### EXAMPLE 11

### (4aSR,11aRS,11bSR)-1-Allyl-1,2,3,4,4a,11,11a,11b-octahydropyrido[2′,3′:3,4]pyrrolo-[1,2-a]indol-5-one

Trifluoroacetic acid is added at ambient temperature to a solution of 80 mg of the compound of Preparation 12 in 3 ml of dichloromethane. The mixture is stirred at ambient temperature for 6 h. The reaction mixture is concentrated under reduced pressure. The residue is taken up in dichloromethane and water. Potassium carbonate is added until the pH of the aqueous phase is basic. After separation of the phases, the aqueous phase, is extracted with dichloromethane. The organic phases are collected, filtered and concentrated under reduced pressure. Chromatography on a silica gel column (cyclohexane/ethyl acetate: 1/1) allows 36 mg of the expected product to be isolated.

Melting point: 163° C.
Mass spectrometry (ES+, m/z): 291 (M+Na)⁺.

### Elemental Microanalyses:

| | C % | H % | N % |
|---|---|---|---|
| Calculated: | 76.09 | 7.51 | 10.44 |
| Found: | 76.00 | 7.61 | 10.37 |

### EXAMPLE 12

### N-(1H-Indol-1-yl)-N′-(3-pyridyl)urea

A solution of 1.14 g of the compound of Preparation 13 in 38 ml of toluene is heated at reflux under argon until the starting material has completely disappeared (2 hours). To the 3-pyridyl isocyanate obtained as intermediate and cooled to 0° C. there are added 995 mg of N-indolamine in 38 ml of dichloromethane. Stirring is continued at ambient temperature for 24 hours. The precipitate formed is filtered off and stirred for 12 hours in water (10 ml). After filtering and washing with pentane, the solid is taken up in a dichloromethane/methanol mixture (90/10). Following filtration, the solid is dried in vacuo over phosphorus pentoxide to yield the expected product.

Melting point. 202.5° C.

### EXAMPLE 13

### N-(2,3-Dihydro-1H-indol-1-yl)-N′-(3-pyridyl)urea

A solution of 942 mg of the compound of Preparation 13 in 30 ml of toluene is heated at reflux under argon until the starting material has completely disappeared (1 hour 30 minutes). The 3-pyridyl isocyanate obtained as intermediate is cooled to 0° C. and 824 mg of 1-indolinamine dissolved in 30 ml of dichloromethane are added. Stirring is then continued at ambient temperature for 15 hours. The precipitate formed is filtered off and washed with diethyl ether, constituting a first batch. The mother liquors are concentrated. The residue is taken up in a minimum of dichloromethane, and diethyl ether is added. Following filtration, the precipitate formed constitutes a second batch, which is recrystallised from ethyl acetate.

Melting point: 197° C.

### Pharmacological Study of Compounds of the Invention

### EXAMPLE A

### Protection from Apoptosis Caused In Vitro in Cultures of Glial Cells

A study was carried out to determine if the compounds of the invention protect cultures of glial cells from the apoptosis brought about by oxygen and glucose deprivation (OGD) followed by reperfusion.

Glial cells are isolated from the brains of newborn Sprague Dawley rats and cultured. They are subjected to OGD for 3 hours by being placed in a glucose-free medium under anaerobic conditions in an incubator (5% CO₂, 95% N₂).

Reperfusion is carried out by returning the cultures for 18 hours to the normal medium [Dulbecco's modified Eagle serum (DMEM)], to which 10% fetal calf serum, 2 mM glutamine and 50 μg/ml of gentamicin have been added.

At the end of the OGD period, the adenosine triphosphate (ATP) content of the cultures is measured in order to assess the degree of ischaemia, using the BIOLUMINESCENT kit (luciferase luminescence).

The degree of apoptosis is quantified by counting the astrocyte nuclei labelled with the DNA-specific HOECHST 33258 fluorescent dye.

The compounds of the invention are added to the medium at a concentration of 10 μM throughout the OGD and reperfusion period.

The results are expressed as the percentage reduction in cell death relative to control cultures subjected to ischaemia by means of OGD and reperfusion.

It was shown that the compounds of the invention have no effect on the levels of ATP: they do not therefore reduce the ischaemia.

In contrast, they greatly reduce the cell death resulting from apoptosis.

TABLE I
Reduction in cell death

| Examples | % reduction |
|---|---|
| Example 1 | 70 |
| Example 4 | 60 |
| Example 6 | 22 |
| Example 7 | 35 |
| Example 9 | 44 |
| Example 10 | 60 |
| Example 11 | 44 |
| Example 12 | 59 |

These results show that the compounds of the invention are capable of protecting glial cells from apoptosis-type cell death without modifying energy metabolism. Hitherto, no substance derived from chemical synthesis has been known to have this effect in vitro.

### EXAMPLE B

### Protection from Neurodegeneration Caused by Ibotenate in Newborn Mice

Ibotenate, an agonist of N-methyl-D-aspartate (NMDA) receptors, when administered by the intracerebral route to the newborn mouse, brings about lesions of excitotoxic origin in the neocortex and the underlying white matter.

The ibotenate injection is performed in newborn mice aged 5 days (P5). The compounds of the invention are administered by the i.p. route at a dose of 20 mg/kg [either simultaneously] (P5), [or 8, 24 or 48 hours after induction of the lesions]. The animals are sacrificed from 24 hours (P6) to 120 hours (P10) after induction of the lesions and the volume of lesions in the cortex and the white matter is measured. Cell markers were applied to the brain slices of animals sacrificed at various times after administration of ibotenate and the compound of the invention: (1) cleaved caspase-3, an apoptosis marker; (2) isolectin, a microglial activation marker; (3) glial fibrillary acidic protein (GFAP), a marker of astrogliosis.

TABLE II
Example 1
Reduction in the size of lesions relative to the controls (%)

| Neuroprotection (simultaneous | Time of sacrifice (h) | | | | |
|---|---|---|---|---|---|
| injection at 0 h) | 8 | 24 | 48 | 72 | 120 |
| cortex | 39 | 40 | 44 | 52 | 46 |
| white matter | 66 | 61 | 52 | 56 | 62 |

| | Delay in | | |
|---|---|---|---|
| | injection after | | |
| Reparatory effect (deferred injection, | ibotenate (h) | | |
| sacrifice at 120 h) | 8 | 24 | 48 |
| cortex | 57 | 39 | 10 |
| white matter | 59 | 61 | 26 |

Reduction in the intensity of markers relative to the controls (%)

| | Time of sacrifice (h) | | | | |
|---|---|---|---|---|---|
| | 8 | 24 | 48 | 72 | 120 |
| Cleaved caspase-3 (apoptosis) | — | 66 | 71 | — | — |
| Isolectin (microglial activation) | 39 | 39 | 35 | 35 | 41 |
| GFAP (astrogliosis) | 24 | 115 | 25 | 31 | 39 |

TABLE III
Reduction in size and intensity
Reduction in the size of lesions (%), simultaneous injection,
sacrifice at 24 and 120 h

| | Example 2 | | Example 3 | | Example 4 | |
|---|---|---|---|---|---|---|
| | 24 h | 120 h | 24 h | 120 h | 24 h | 120 h |
| Cortex | — | 63 | 53 | 50 | NS | NS |
| White matter | — | 38 | 70 | 56 | NS | NS |

Reduction in the size of lesions (%), simultaneous injection,
sacrifice at 24 and 120 h

| | Example 9 | | Example 12 | | Example 13 | |
|---|---|---|---|---|---|---|
| | 24 h | 120 h | 24 h | 120 h | 24 h | 120 h |
| Cortex | — | 50 | — | 64 | — | 28 |
| White matter | — | 56 | — | 58 | — | 11 |

Reduction in the intensity of markers relative to the controls (%)

| | Example 2 | | Example 3 | | Example 4 | |
|---|---|---|---|---|---|---|
| | 24 h | 120 h | 24 h | 120 h | 24 h | 120 h |
| Cleaved caspase-3 (apoptosis) | 51 | — | 56 | — | NS | — |
| Isolectin | 50 | — | 50 | — | NS | — |
| (microglial activation) | | | | | | |
| GFAP (astrogliosis) | — | 46 | | 44 | | NS |

In addition, the reparatory effect was tested for Example 2, which also shows a reduction in the size of the lesions of 27% for the cortex and 37% for the white matter when it is injected 24 hours after ibotenate, when the lesions have already developed.

### EXAMPLE C

### Effect of the Compounds of the Invention on Cell Differentiation of Stem Cells

Mouse embryonic stem (ES) cells are isolated from the internal cell mass of the blastocyst. Depending on the culture conditions, they can multiply indefinitely or can differentiate to give rise to various types of cell. The ES cells differentiate into neural precursors which, in turn, develop into a heterogeneous population of cells composed of astrocytes, oligodendrocytes and different types of neurons (GABAergic, glutamatergic, dopaminergic, cholinergic, glycinergic etc.) (Okabe S., Forsberg-Nilsson K., Spiro A. C. et al., Mechanisms of Development, (1996), 59, 89-102; Brüstle O., Spiro A. C., Karram K. et al., Proc Natl Acad Sci USA, (1997), 94, 14809-14814; Guan K., Chang, Rolletschek A. et al., Cell Tissu Res, (2001), 305, 171-176). Adding certain differentiation inducers such as retinoic acid (RA) to the culture medium facilitates orientation of the ES cells towards the neuronal route (Strübing C., Ahnert-Hilger G., Shan J. et al., Mechanisms of Development, (1995), 53, 275-287). The obtaining of a homogeneous population of neurons constitutes a key step in the objective of treating neurodegenerative diseases whether on the basis of transplanting ES or for differentiation of the ES of the patient.

The experiment is carried out in the following manner: 12 hours after dividing the confluent ES cells for subculturing, they are treated with compounds of the invention at concentrations of 0.1; 1 and 10 μM and with RA (1 μM). The treatment is repeated every 2 days and the cell extracts are made 3 hours after each treatment, on the 1st day (3 hours), the 4th day (D4) and 8th day (D8) for quantification of the markers by Q-RT-PCR -(quantitative reverse transcription polymerase chain reaction).

These markers are: nestin, neural precursor marker; synaptophysin, neuronal differentiation and synaptic plasticity marker; TH, marker of differentiation into neurons of dopaminergic or noradrenergic type; glutamic acid decarboxylase (GAD), marker of differentiation of ES into GABAergic neurons; GFAP, specific astrocyte marker.

After 8 days of exposure, Example 2 at 1 μM increases synaptophysin (229±37%; p<0.05), TH (253±62%) and GFAP (67%), indicating that Example 2 is capable of bringing about, starting from embryonic stem cells, the appearance of a neuronal phenotype and the neuronal precursors differentiate preferentially into dopaminergic or noradrenergic, and not GABA-ergic, neurons. In parallel, Example 2 allows differentiation of the stem cells into glial cells, which are necessary to the survival of the neuron.

### EXAMPLE D

### Effect of Compounds of the Invention on Glial Reaction in a Model of Parkinson 's Disease in the Mouse

Unilateral injection of 6-OHDA into the mouse striatum using a micro-syringe (0.5 μl of a solution containing 8 μg/μl) causes, from the 3rd day, degeneration of the TH-positive neurons of the striatum, which is accompanied, from the 7th day, by neuronal loss also in the substantia nigra, in accordance with that which is observed. in Parkinson's disease in humans. The degeneration of dopaminergic neurons is accompanied by glial hyper-reactivity, demonstrated by the increase in cells marked with GFAP.

The compounds of the invention are administered by the intraperitoneal route in a single injection of 20 mg/kg 10 minutes after the intracerebral injection of 6-OHDA. One group of mice is sacrificed at 3 days, the other at 7 days.

The area occupied by astrocytes, which is marked with GFAP, is significantly increased in the striatum of the damaged side, reflecting the astrocyte reaction.

A single injection of Example 1 reduces by 13% the area occupied by the astrocytes in the striatum, assessed at 7 days, relative to the control group which received 6-OHDA and the solvent.

A single injection of Example 3 reduces by 28% the area occupied by the astrocytes in the striatum, assessed at 7 days, relative to the control group which received 6-OHDA and the solvent, with virtual normalisation compared to the healthy animals being observed.

In parallel thereto, under the effect of Example 3, the microglial reactivity remains increased at 3 days, thereby allowing the microglia to play their part in removal of the debris, and is then reduced at 7 days thereby bringing the damaged striatum back towards that of the healthy animals.

This experiment suggests that the compounds of the invention are capable of protecting against neurodegeneration in neurodegenerative diseases such as Parkinson's disease. These properties might extend to other degenerative pathologies such as Alzheimer's disease, Huntington's disease, amyotrophic lateral sclerosis, multiple sclerosis, pathological ageing, cerebral vascular accident and also psychiatric disorders such as depression, schizophrenia and senile dementias.

### EXAMPLE E

### Neuroprotective and Neurotrophic Effects of Compounds in an In Vitro Model of Neurodegeneration

The study is carried out on mixed (neurons and glia) primary embryonic mesencephalic cultures, in the presence or not of a neurotoxic inducer, epoxomicin.

The cells are obtained from embryos of female rats at the 14th day of gestation. The mesencephalon is dissected, the tissues removed and dissociated using trypsin and then mechanically. The cells, which are viable, are seeded onto 96-well plates and horse serum is added.

The compounds are added at concentration ranging from 100 nM to 10 μM in the presence, or not, of epoxomicin (50 nM). Epoxomicin is a potent selective proteasome inhibitor known to induce cell-death via apoptotic mechanisms. Inhibition of the ubiquitin-proteasome complex also reproduces the characteristics of Parkinson's disease.

After 48 hours, the cells are detached, fixed and labelled with an antibody that recognises the NeuN protein expressed by all of the cell population (neurons and glia).

The results are expressed as a percentage of the control value, in the presence or absence of epoxomicin.

After 48 hours, epoxomicin causes neuronal loss of 82% relative to the untreated cells and a loss, over all of the cell population, of slightly less than 70% relative to the cells not treated with epoxomicin.

The compounds of the invention tested in the absence of epoxomicin have a neurotrophic effect if they increase neuronal survival. In the presence of epoxomicin, it is their neuroprotective power which is demonstrated when the compounds increase neuronal survival.

Some compounds have both neuroprotective and neurotrophic properties; others have either one or the other.

### Results

Neuroprotective and neurotrophic compounds: Example 2 doubles neuronal survival between 100 nM and 30 μM, in the absence of epoxomicin (neurotrophic). At concentrations greater than or equal to 10 μM, it has neuroprotective properties.

Neuroprotective compounds: Example 8 and Example 13 bring about an increase in neuronal survival of the order of 75 and 145%, respectively, with respect to their control, at 100 μM.

Neurotrophic compounds: Example 9 is neurotrophic, with an EC₅₀ of 10.5 μM.

### EXAMPLE F

### Neuroprotective Effects and Stimulation of the Catecholaminergic Phenotype In Vitro

The study is carried out in an in vitro model of primary rat mesencephalic cultures, wherein death of the dopaminergic neurons develops spontaneously and selectively when the cells mature. The culture conditions have been defined in detail in various publications (Douhou et al., J. of Neurochemistry, (2001), 78, 163-174). The culture media are changed daily and, under these conditions, the dopaminergic neurons, but not the other neurons such as GABA-ergic neurons, degenerate spontaneously. Natural trophic factors of the body such as GDNF (glial cell derived neurotrophic factor) have shown a protective effect on the dopaminergic neurons in this model.

The effects of Examples 1 and 12 were tested at 3 concentrations. The effect is analysed by measuring the rate of tritiated dopamine uptake and the number of surviving neurons by labelling the tyrosine hydroxylase with a fluorescent antibody. At 30CM, Examples 1 and 12 significantly increase cell survival and the rate of tritiated dopamine uptake.

### Discussion

Examples A (in vitro) and B (in vivo) show that the compounds of the invention exert their protection at the level of the microglia. Glial activation is, it should be noted, the common denominator in neurodegenerative diseases.

Microglial activation is the early response. by the central nervous system to a variety of pathogenic stimuli, whether traumatic, inflammatory, degenerative or ischaemic (Ito D., Tanaka K., Suzuki S. et al., Stroke, (2001), 32, 1208-1215). The glial cells exert a beneficial effect by removing the harmful debris and by secreting neurotrophic factors and, at the same time, a cytotoxic effect by releasing oxygen free radicals or inflammatory cytokines responsible for apoptosis of the neurons or astrocytes (Smith M. E., van der Maesen K., Somera F. P., J Neurosci Res, (1998), 54, 68-78).

Example D very especially demonstrates that compounds of the invention are capable of reducing the reactivity of the microglia at 7 days in a model of Parkinson's disease whereas at 3 days the non-modified activation of the microglia allows them to exert their beneficial effect.

Example B moreover shows that the compounds of the invention equally protect grey matter, that is to say the neuronal cell bodies and their dendrites, and white matter, essentially formed of myelinated and non-myelinated fibres, and grey cells. Accordingly the pathologies targeted include disorders affecting both the grey matter and the white matter.

Finally, the compounds of the invention are capable not only of limiting neuronal death (Examples A, B, D, E and F) but, starting from embryonic stem cells of the species in question, they bring about the appearance of a neuronal phenotype of the dopaminergic or noradrenergic type as well as the differentiation of stem cells into the glial cells necessary for the survival of the neurons (Example C).

Accordingly, the compounds of the invention claim, on the basis of a common mechanism, a very wide spread of neurodegenerative pathologies originating from a stimulus that is neurodegenerative, traumatic, inflammatory, viral, ischaemic, genetic or excitotoxic or a stress or from defects of neurogenesis, including Alzheimer's disease, earlier forms of dementia such as MCI , Parkinson's disease, Huntington's disease, multiple sclerosis, motor neuron diseases such as amyotrophic lateral sclerosis, defects of cerebral perfusion such as cerebral vascular accident of thrombotic origin, haemorrhagic origin or following cardiac surgery, epilepsy, virus diseases (HIV) or prion diseases, all phenomena-of neuroplasticity. found in psychiatric disorders: autism (Wickelgren I., Science, (2005), 308, 1856-1558), dyslexia, schizophrenia, depression, attention-deficit hyperactivity (ADHD) and also all pathologies of the white matter such as the lesions of periventricular leukomalacia of premature infants, atrophy of the cerebral white matter associated with ageing, with hypertension and leading to dementia, and the lesions of leukoaraiosis observed in chronic arterial hypertension, especially in elderly patients.

### EXAMPLE G

### Pharmaceutical Composition

Formula for the preparation of 1000 tablets each containing 10 mg of active ingredient

| | | |
|---|---|---|
| Compound of Example 1 | 10 | g |
| Hydroxypropylcellulose | 2 | g |
| Wheat starch | 10 | g |
| Lactose | 100 | g |
| Magnesium stearate | 3 | g |
| Talc | 3 | g |

1-10. (canceled)

11- A method for treating and/or preventing neurodegenerative diseases and disorders resulting from neurodegenerative diseases, such method comprising the step of administering to a living animal, including a human, a therapeutically effective amount of a compound selected from those of formula (II):
wherein: 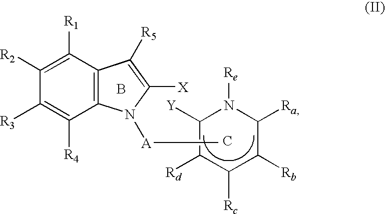
A represents a divalent radical: 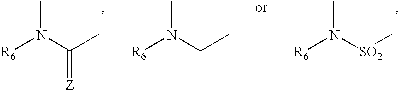
wherein:
Z represents an oxygen atom or sulphur atom,
R₆ represents a hydrogen atom, a linear or branched (C₁-C₆)alkyl group, C(O)-AA wherein AA represents an amino acid radical, a linear or branched (C₁-C₆)alkoxy-carbonyl group, CHR′—O—C(O)—R″ wherein R′ represents a hydrogen atom or a linear or branched (C₁-C₆)alkyl group and R″ represents a linear or branched (C₁-C₆)alkyl group, a linear or branched (C₂-C₆)alkenyl group, an aryl group, an aryl-(C₁-C₆)alkyl group in which the alkyl moiety is linear or branched, a linear or branched (C₁-C₆)polyhaloalkyl group, or a linear or branched (C₁-C₆)alkyl chain substituted by one or more halogen atoms, one or more hydroxy groups, linear or branched (C₁-C₆)alkoxy groups, or amino groups optionally substituted by one or two identical or different, linear or branched (C₁-C₆)alkyl groups,
in the ring B [symbol]
represents a single bond or a double bond,
in the ring C [symbol]
represents a single bond or a double bond, the ring C containing, at most, only one double bond,
R₁, R₂, R₃ and R₄, which may be the same or different, each independently of the others, represent a hydrogen atom, a halogen atom, a linear or branched (C₁-C₆)alkyl group, a linear or branched (C₁-C₆)alkoxy group, a hydroxy group, a cyano group, a nitro group, a linear or branched (C₁-C₆)polyhaloalkyl group, an amino group (optionally substituted by one or two linear or branched (C₁-C₆)alkyl and/or linear or branched (C₂-C₆)alkenyl groups, it being possible for the alkyl and alkenyl groups to be the same or different), or a linear or branched (C₁-C₆)alkyl chain substituted by one or more halogen atoms, one or more hydroxy groups, linear or branched (C₁-C₆)alkoxy groups, or amino groups optionally substituted by one or two identical or different, linear or branched (C₁-C₆)alkyl groups,
R₅ represents a hydrogen atom, a linear or branched (C₁-C₆)alkyl group, an aminoalkyl group in which the alkyl moiety is a linear or branched chain of 1 to 6 carbon atoms, or a linear or branched (C₁-C₆)hydroxyalkyl group,
X and Y, which may be the same or different, each independently of the other, represent a hydrogen atom or a linear or branched (C₁-C₆)alkyl group,
Rₐ, R_{b}, R_{c} and R_{d}, which may be the same or different, each independently of the others, represent a hydrogen atom, a halogen atom, a linear or branched (C₁-C₆)alkyl group, a hydroxy group, a linear or branched (C₁-C₆)alkoxy group, a cyano group, a nitro group, a linear or branched (C₁-C₆)polyhaloalkyl group, an amino group (optionally substituted by one or two identical or different, linear or branched (C₁-C₆)alkyl groups), or a linear or branched (C₁-C₆)alkyl chain substituted by one or more groups selected from halogen, hydroxy, linear or branched (C₁-C₆)alkoxy, and amino optionally substituted by one or two identical or different, linear or branched (C₁-C₆)alkyl groups,
it being understood that when A is linked to the ring C at a carbon atom carrying one of the substituents Rₐ, R_{b}, R_{c}, R_{d} or Y and said linking carbon atom also carries a double bond, then the corresponding substituent Rₐ, R_{b}, R_{c}, R_{d} or Y is absent,
Rₑ represents a hydrogen atom, a linear or branched (C₁-C₆)alkyl group; an aryl-(C₁-C₆)alkyl group in which the alkyl moiety is linear or branched; a linear or branched (C₂-C₆)alkenyl group; a linear or branched (C₂-C₆)alkynyl group; a linear or branched (C₁-C₆)alkyl chain substituted by one or more groups selected from hydroxy, amino (optionally substituted by one or two identical or different, linear or branched (C₁-C₆)alkyl groups), linear or branched (C₁-C₆)alkoxy, and NR₇R₈ wherein R₇ and R₈, together with the nitrogen atom carrying them, form an optionally substituted, 4- to 8-membered heterocycle optionally containing one or more double bonds within the heterocycle and optionally containing within the cyclic system a second hetero atom selected from an oxygen atom and a nitrogen atom; or a linear or branched (C₂-C₆)alkenyl chain substituted by the same groups as the alkyl chain or a linear or branched (C₂-C₆)alkynyl chain substituted by the same groups as the alkyl chain,
its enantiomers, diastereoisomers, and addition salts thereof with a pharmaceutically acceptable acid or base.

12- The method of claim 11, wherein the neurodegenerative disease is selected from neurodegenerative pathologies originating from a stimulus that is neurodegenerative, traumatic, inflammatory, viral, ischaemic, genetic or excitotoxic or a stress or from defects of neurogenesis, including Alzheimer's disease; earlier forms of dementia including MCI ; Parkinson's disease, Huntington's disease, multiple sclerosis, motor neuron diseases including amyotrophic lateral sclerosis; pathological aging; defects of cerebral perfusion including cerebral vascular accident of thrombotic origin, haemorrhagic origin or following cardiac surgery; epilepsy; virus diseases including HIV; prion diseases; cerebral or spinal cord trauma; all phenomena of neuroplasticity found in psychiatric disorders: autism, dyslexia, schizophrenia, depression, attention-deficit hyperactivity (ADHD); and all pathologies of the white matter including lesions of periventricular leukomalacia of premature infants, atrophy of the cerebral white matter associated with aging, with hypertension and leading to dementia, and lesions of leukoaraiosis.

13- A method for treating and/or preventing neurodegenerative diseases and disorders resulting from neurodegenerative diseases, such method comprising the step of administering to a living animal, including a human, a therapeutically effective amount of a compound selected from those of formula (III): 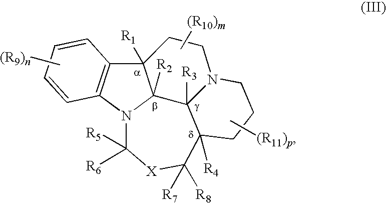
wherein:
R₁ represents a hydrogen atom, a linear or branched (C₁-C₆)alkyl group, a linear or branched (C₁-C₆)aminoalkyl group or a linear or branched (C₁-C₆)hydroxyalkyl group,
R₂ represents a hydrogen atom,
or R₁ and R₂, together with the carbon atoms carrying them, form a carbon-carbon bond,
R₃ represents a hydrogen atom,
R₄ represents a hydrogen atom, a methyl group, a linear or branched (C₃-C₆)alkyl group, a linear or branched (C₁-C₆)aminoalkyl group, a linear or branched (C₁-C₆)hydroxyalkyl group, an aryl-(C₁-C₆)alkyl group in which the alkyl moiety is linear or branched, or a heterocycloalkyl-(C₁-C₆)alkyl group in which the alkyl moiety is linear or branched,
or R₃ and R₄, together with the carbon atoms carrying them, form a carbon-carbon bond,
R₅, R₆, R₇ and R₈, which may be identical or different, represent, each independently of the others, a hydrogen atom
or a pair of geminal substituents (R₅ and R₆ and/or R₇ and R₈) form an oxo, thioxo or imino group,
R₉ represents a hydrogen atom, a halogen atom, an optionally substituted, linear or branched (C₁-C₆)alkyl group, a linear or branched (C₁-C₆)alkoxy group, a hydroxy group, a cyano group, a nitro group, a linear or branched (C₁-C₆)polyhaloalkyl group or an amino group (optionally substituted by one or two linear or branched (C₁-C₆)alkyl(s), linear or branched (C₂-C₆)alkenyl(s), wherein the alkyls and alkenyls may be identical or different),
R₁₀ and R₁₁, which may be identical or different, represent, each independently of the other, a hydrogen atom, a halogen atom, a linear or branched (C₁-C₆)alkyl group, a linear or branched (C₁-C₆)alkoxy group, a hydroxy group, a cyano group, a nitro group, a linear or branched (C₁-C₆)polyhaloalkyl group or an amino group (optionally substituted by one or two linear or branched (C₁-C₆)alkyl(s), linear or branched (C₂-C₆)alkenyl(s), it being possible for the alkyls and alkenyls to be identical or different),
n represents an integer between 0 and 4 inclusive,
m represents an integer between 0 and 2 inclusive,
p represents an integer between 0 and 3 inclusive,
X represents a group NR₁₂,
R₁₂ represents a hydrogen atom, an optionally substituted, linear or branched (C₁-C₆)alkyl group, an optionally substituted, linear or branched (C₂-C₆)alkenyl group, an aryl-(C₁-C₆)alkyl group in which the alkyl moiety is linear or branched, or a linear or branched (C₁-C₆)polyhaloalkyl group,
its enantiomers, diastereoisomers, and addition salts thereof with a pharmaceutically acceptable acid or base.

14- The method of claim 13, wherein the neurodegenerative disease is selected from neurodegenerative pathologies originating from a stimulus that is neurodegenerative, traumatic, inflammatory, viral, ischaemic, genetic or excitotoxic or a stress or from defects of neurogenesis, including Alzheimer's disease; earlier forms of dementia including MCI; Parkinson's disease, Huntington's disease, multiple sclerosis, motor neuron diseases including amyotrophic lateral sclerosis; pathological aging; defects of cerebral perfusion including cerebral vascular accident of thrombotic origin, haemorrhagic origin or following cardiac surgery; epilepsy; virus diseases including HIV; prion diseases; cerebral or spinal cord trauma; all phenomena of neuroplasticity found in psychiatric disorders: autism, dyslexia, schizophrenia, depression, attention-deficit hyperactivity (ADHD); and all pathologies of the white matter including lesions of periventricular leukomalacia of premature infants, atrophy of the cerebral white matter associated with aging, with hypertension and leading to dementia, and lesions of leukoaraiosis.

15- A method for treating and/or preventing neurodegenerative diseases and disorders resulting from neurodegenerative diseases, such method comprising the step of administering to a living animal, including a human, a therapeutically effective amount of a compound selected from those of formula (IV): 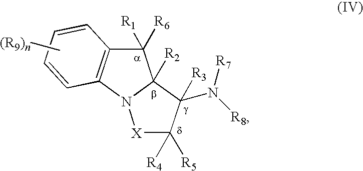
wherein:
X represents CO or 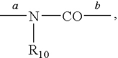
R₁ represents hydrogen, linear or branched (C₁-C₆)alkyl, linear or branched (C₁-C₆)aminoalkyl, linear or branched (C₁-C₆)hydroxyalkyl, aryl-(C₁-C₆)alkyl in which the alkyl moiety may be linear or branched,
R₂ represents hydrogen, linear or branched (C₁-C₆)alkyl, linear or branched (C₁-C₆)aminoalkyl, linear or branched (C₁-C₆)hydroxyalkyl,
or R₁ and R₂, together with the carbon atoms carrying them, form a carbon-carbon bond,
R₃ represents hydrogen, linear or branched (C₁-C₆)alkyl, linear or branched (C₁-C₆)aminoalkyl, linear or branched (C₁-C₆)hydroxyalkyl,
R₄ represents hydrogen, linear or branched (C₁-C₆)alkyl, linear or branched (C₁-C₆)aminoalkyl, linear or branched (C₁-C₆)hydroxyalkyl,
or R₃ and R₄, together with the carbon atoms carrying them, form a carbon-carbon bond,
R₅ represents hydrogen, linear or branched (C₁-C₆)alkyl,
R₆ represents hydrogen, linear or branched (C₁-C₆)alkyl,
R₇ and R₈ represent hydrogen, linear or branched (C₁-C₆)alkyl, aryl-(C₁-C₆)alkyl in which the alkyl moiety may be linear or branched, linear or branched (C₂-C₆)alkenyl, linear or branched (C₂-C₆)alkynyl, a linear or branched (C₁-C₆)alkyl chain substituted by one or more substituents selected from hydroxy, cyano, linear or branched (C₁-C₆)alkoxy, and NR₁₃R₁₄, a linear or branched (C₁-C₆)alkenyl chain substituted by one or more substituents selected from hydroxy, cyano, linear or branched (C₁-C₆)alkoxy, and NR₁₃R)₄, or a linear or branched (C₁-C₆)alkynyl chain substituted by one or more substituents selected from hydroxy, cyano, linear or branched (C₁-C₆)alkoxy, and NR₁₃R₁₄,
or,
either R₅ and R₈, together with the carbon and nitrogen atoms carrying them, form a heterocycle having 5, 6 or 7 ring members, optionally substituted by a group R₁₂, or R₆ and R₇, together with the carbon and nitrogen atoms carrying them, form a heterocycle having 5, 6 or 7 ring members, optionally substituted by a group R₁₁,
wherein only one of the two groupings “R₅ and R₈” or “R₆ and R₇” together with the carbon and nitrogen atoms carrying them may form a heterocycle having 5, 6 or 7 ring members, optionally substituted by a group R₁₁,
R₉ represents hydrogen, halogen, linear or branched (C₁-C₆)alkyl, linear or branched (C₁-C₆)alkoxy, hydroxy, cyano, nitro, linear or branched (C₁-C₆)polyhaloalkyl, NR₁₅R₁₆, or a linear or branched (C₁-C₆)alkyl chain substituted by one or more halogens, one or more hydroxy, linear or branched (C₁-C₆)alkoxy, or NR₁₅R₁₆,
n represents an integer 0, 1, 2, 3 or 4,
R₁₀ represents hydrogen, linear or branched (C₁-C₆)alkyl, linear or branched (C₂-C₆)alkenyl, aryl-(C₁-C₆)alkyl in which the alkyl moiety may be linear or branched, linear or branched (C₁-C₆)polyhaloalkyl, or a linear or branched (C₁-C₆)alkyl chain substituted by one or more halogen atoms, one or more hydroxy groups, linear or branched (C₁-C₆)alkoxy groups, or NR₁₅R₁₆ groups,
R₁₁, R₁₂, which may be identical or different, represent a —COOT or —CH₂O-U group wherein T and U, which may be identical or different, represent a hydrogen atom or a linear or branched (C₁-C₆)alkyl group,
R₁₃, R₁₄, which may be identical or different, represent a hydrogen atom or a linear or branched (C₁-C₆)alkyl group or, together with the nitrogen atom carrying them, form an optionally substituted heterocycle having from 4 to 8 ring members optionally containing a double bond in the heterocycle and optionally containing in the ring system a second hetero atom selected from an oxygen atom and a nitrogen atom,
R₁₅, R₁₆, which may be identical or different, each represent a hydrogen atom or a group linear or branched (C₁-C₆)alkyl, linear or branched (C₂-C₆)alkenyl,
its enantiomers, diastereoisomers, and addition salts thereof with a pharmaceutically acceptable acid or base,
it being understood that the compound of formula (IV) may not represent:(3aSR,4SR)-3-benzyl-4-ethyl-2,3,3a,4-tetrahydrobenzo[b]pyrido[2,3,4-gh]pyrrolizin-5(1H)-one.

16- The method of claim 15, wherein the neurodegenerative disease is selected from neurodegenerative pathologies originating from a stimulus that is neurodegenerative, traumatic, inflammatory, viral, ischaemic, genetic or excitotoxic or a stress or from defects of neurogenesis, including Alzheimer's disease; earlier forms of dementia including MCI ; Parkinson's disease, Huntington's disease, multiple sclerosis, motor neuron diseases including amyotrophic lateral sclerosis; pathological aging; defects of cerebral perfusion including cerebral vascular accident of thrombotic origin, haemorrhagic origin or following cardiac surgery; epilepsy; virus diseases including HIV; prion diseases; cerebral or spinal cord trauma; all phenomena of neuroplasticity found in psychiatric disorders: autism, dyslexia, schizophrenia, depression, attention-deficit hyperactivity (ADHD); and all pathologies of the white matter including lesions of periventricular leukomalacia of premature infants, atrophy of the cerebral white matter associated with aging, with hypertension and leading to dementia, and lesions of leukoaraiosis.

17- A method for treating and/or preventing neurodegenerative diseases and disorders resulting from neurodegenerative diseases, such method comprising the step of administering to a living animal body, including a human, a therapeutically effective amount of a compound selected from those of formula (V): 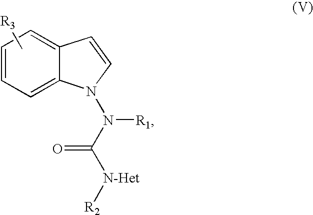
wherein:
R₁ and R₂, which may be the same or different, represent a hydrogen atom or a linear or branched (C₁-C₆)alkyl group,
R₃ represents a hydrogen or halogen atom, a linear or branched (C₁-C₆)alkyl group, or a linear or branched (C₁-C₆)alkoxy group,
Het represents a pyridyl, pyrimidinyl or piperidyl group, optionally substituted by one or more groups selected from halogen, linear or branched (C₁-C₆)alkyl and linear or branched (C₁-C₆)alkoxy, [symbol]
represents a single bond or a double bond,
it being understood that R₃ may be attached to any of the carbons of the indole/indoline nucleus that allows it,
its enantiomers, diastereoisomers, and addition salts thereof with a pharmaceutically acceptable acid or base.

18- The method of claim 17, wherein the neurodegenerative disease is selected from neurodegenerative pathologies originating from a stimulus that is neurodegenerative, traumatic, inflammatory, viral, ischaemic, genetic or excitotoxic or a stress or from defects of neurogenesis, including Alzheimer's disease; earlier forms of dementia including MCI ; Parkinson's disease, Huntington's disease, multiple sclerosis, motor neuron diseases including amyotrophic lateral sclerosis; pathological aging; defects of cerebral perfusion including cerebral vascular accident of thrombotic origin, haemorrhagic origin or following cardiac surgery; epilepsy; virus diseases including HIV; prion diseases; cerebral or spinal cord trauma; all phenomena of neuroplasticity found in psychiatric disorders: autism, dyslexia, schizophrenia, depression, attention-deficit hyperactivity (ADHD); and all pathologies of the white matter including lesions of periventricular leukomalacia of premature infants, atrophy of the cerebral white matter associated with aging, with hypertension and leading to dementia, and lesions of leukoaraiosis.
